(19) United States
(12) Patent Application Publication
(10) Pub. No.: US 2023/0026360 A1
(43) Pub. Date: Jan. 26, 2023

(54) DETECTION OF MOLECULAR INTERACTIONS

(71) Applicants: Orionis Biosciences, Inc., Waltham, MA (US); Orionis Biosciences BV, Ghent (BE)

(72) Inventors: Nikolai KLEY, Waltham, MA (US); Samuel LIEVENS, Ghent (BE); Edward SUH, Waltham, MA (US)

(21) Appl. No.: 17/785,359

(22) PCT Filed: Dec. 15, 2020
(86) PCT No.: PCT/US20/65024

§ 371 Date: Jun. 14, 2022

Related U.S. Application Data
(60) Provisional application No. 62/949,023, filed on Dec. 17, 2019.

(51) Int. Cl. C12N 15/10

(52) U.S. Cl. CPC C12N 15/1055

(57) ABSTRACT

Methods for detection of molecular interactions, such as protein/protein or small molecule/protein interactions, are described.

### CROSS-REFERENCE TO RELATED APPLICATIONS

This application claims the benefit of U.S. Provisional Application No. 62/949,023, filed on Dec. 17, 2019, the entire contents of which are incorporated herein.

### FIELD

The present invention is related to, inter alia, detection and identification protein-protein or protein-small molecule interactions, and/or novel small molecules.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (Filename: “ORN-060PC_ST25.txt”; Date created: Dec. 7, 2020; File size: 10,349 bytes).

### BACKGROUND

Molecular interactions, such as protein/protein and protein/small molecule interactions, are a key part of many, if not all, biological processes. Several approaches have been developed with the objective of identifying molecular interactions. For instance, biochemical approaches include co-purification, co-immunoprecipitation, among others. However, these techniques are tedious, do not allow high throughput screening, and require lysis, which corrupts the normal cellular context. Genetic approaches solve some of these problems. For instance, yeast two-hybrid methods have displayed excellent utility. Despite widespread adoption, however, the yeast two-hybrid system has several drawbacks, including requiring that the fusion proteins be translocated to the nucleus. Another approach is phage display, which does not require nuclear translocation. However, the phage display approaches suffer from an artificiality—i.e., the proteins need to be exposed at the phage surface where the proteins are exposed to an environment that may not be physiologically relevant, which hinders one's ability to equate with the interactions of a living cell.

Thus, there remains a need for new methods for detecting molecular interactions.

### SUMMARY

Accordingly, the present invention relates, in part, to a cell-based system for detecting various molecular interactions. In some embodiments, the present invention provides for methods that allow interrogation of molecular interactions (e.g., protein/protein, protein/small molecule, and/or protein/protein interactions that are modulated by small molecules) which are not detectable using standard assays. For instance, the present methods, in some embodiments, reduce or eliminate instances of false positive or false negative signals.

In some embodiments, the present methods employ an inversion of bait and prey relative to known methods, such as cytokine-receptor-based interaction trap methods, and as described herein, that allows for improved molecular interaction detection.

For example, as described herein, the “forward” version of the Mammalian protein-protein interaction trap (MAPPIT, see Eyckerman, et al. “Design and application of a cytokine-receptor-based interaction trap,” Nat Cell Biol. 2001 December; 3(12):1114-9 and Lievens, et al. “Proteome-scale Binary Interactomics in Human Cells,” Molecular & Cellular Proteomics 15.12 (2016): 3624-3639, incorporated by reference in their entireties) can suffer from deficiencies in certain instances. Various embodiments of the present invention cure these deficiencies, for example, by inverting the method design. For instance, in various embodiments, the present methods allow for detection and/or discovery of interactions that are not clearly identified, e.g. with the forward MAPPIT method (described herein) because, for instance, an interacting partner is sequestered in the nucleus or/an organelle of the cell when expressed in the forward MAPPIT assay format. By way of further example, in some embodiments, the present methods allow for detection of interactions that are not clearly identified with the forward MAPPIT method because an interacting partner expressed in that assay format contacts the membrane of the cell in a non-specific way and/or contacts the membrane-based construct being used for the present detection in a non-specific way when expressed in the forward MAPPIT assay format.

In various embodiments, the present invention relates to a method for detecting a molecular interaction by (a) providing a cell having a ligand-based chimeric receptor having (i) an extracellular portion of a ligand-binding domain derived from a first receptor and (ii) transmembrane and cytoplasmic domains of a second receptor and having an intracellular prey protein fused thereto, wherein the transmembrane and/or cytoplasmic domains of the second receptor comprise mutations that reduce or eliminate STAT (Signal Transducer and Activator of Transcription) recruitment; (b) expressing a bait protein that is fused to a receptor fragment in the cell, the receptor fragment comprising functional STAT recruitment sites; and (c) detecting a signal that is indicative of a molecular interaction wherein the bait protein (i) favors occupying the cytosol over the interior of a membrane bound organelle and/or (ii) favors specific interaction with the prey protein over non-specific interaction with the cell membrane and/or a non-prey portion of the chimeric receptor. In embodiments, the first receptor and second receptor are the same.

In some embodiments, the interaction between the prey protein and bait protein causes recruitment of the receptor fragment to the cytoplasmic domain of the second receptor that is fused to a first receptor, which restores ligand-dependent receptor signaling and activation of STAT molecules. In some embodiments, the cell comprises a STAT-responsive reporter gene. In some embodiments, the activated STAT molecules migrate to the nucleus and induce transcription of a STAT-responsive reporter gene and, in some instances, the reporter gene signal permits detection of a molecular interaction.

In some embodiments, the molecular interaction is a protein/protein interaction. In some embodiments, the molecular interaction is a protein/protein interaction, which is mediated by a small molecule (e.g., the method further comprises introducing a small molecule which binds to the prey protein or bait protein). Specifically, in some embodiments, the molecular interaction is a protein/protein interaction, which is mediated by the binding of the small molecule with the prey protein or bait protein. For example, the present methods may detect a complex formation. In some embodiments, the small molecule induces exposure of a hydrophobic surface of the prey protein or bait protein that allows for interaction with the prey protein or bait protein. In some embodiments, the small molecule is a molecular glue or a bivalent hybrid ligand molecule (e.g., without limitation a PROTAC).

By way of example, in some embodiments, the interactions detected involve an E3 ligase protein, e.g., without limitation, in contact with an Immunomodulatory Drug (IMiD) e.g., thalidomide, lenalidomide and pomalidomide, and compounds related thereto and/or compounds that bind to the same or similar site (pocket) in the cereblon (CRBN) protein, which, in some embodiments, is the bait fused to (or indirectly bound to) the receptor fragment in the present “Inverse” assay.

### BRIEF DESCRIPTION OF THE DRAWINGS

FIG. 1 shows the MAPPIT concept. A bait protein (“B”) is C-terminally fused to a chimeric receptor having the extracellular part of a type I cytokine receptor (“CYT”) and the transmembrane and intracellular domains of a receptor that is made deficient in STAT recruitment via mutagenesis. When co-expressed with a prey protein (“P”) that is fused to a receptor fragment containing functional STAT recruitment sites, the receptor complex is functionally complemented and, upon cytokine ligand stimulation (L), signaling is restored. STAT molecules are activated and migrate to the nucleus and induce transcription of a STAT-responsive reporter gene. Throughout this disclosure, this modality is referred to as the “Forward” assay.

FIG. 2A outlines a deficiency of the system of FIG. 1, namely the creation of a false negative signal. Here, the prey is unable to reach the bait fused to the membrane construct, e.g., by being sequestered in the nucleus and/or an organelle (left panel). The right panel shows an inversion of the bait and prey to free the prey from sequestration, which allows for detection of a molecular interaction (throughout this disclosure, this modality is referred to as the “Inverse” assay).

FIG. 2B outlines a further deficiency of the system of FIG. 1, namely another example of the creation of a false negative signal. Here, the prey is interacting in an unspecific way to the membrane construct (left panel). The right panel shows an inversion of the bait and prey to offset/mitigate the unspecific interaction (right panel, throughout this disclosure, this modality is referred to as the “Inverse” assay).

FIG. 3 outlines an alternative construction of the “Inverse” assay. In this construction, the bait, which can interact with the small molecule and/or prey, is associated with a scaffold protein. The cross-hatched segment is the scaffold protein that is expressed independently in the cell or as a direct fusion with the bait protein.

FIGS. 4A-F Evaluation of CRBN-binding compounds recruiting selected substrates in MAPPIT forward and/or inverse assay configuration. Recruitment induced by lenalidomide and CC-220 CRBN IMiD ligands of a panel of known CRBN substrates described in the literature was evaluated in MAPPIT, a variation of a two-hybrid technology system described previously (Lemmens, et al. “MAPPIT, a mammalian two-hybrid method for in-cell detection of protein-protein interactions,” Methods Mol Biol. 2015; 1278:447-55, the entire contents of which are herein incorporated by reference) and outlined in more detail in Example 1. More specifically, two configurations of the MAPPIT assay were compared, referred to as ‘forward’ and ‘inverse’ assay modes, which differ in whether the bait protein, in this case CRBN, is fused to the MAPPIT chimeric transmembrane receptor (in the classical ‘forward’ mode) or the soluble gp130 receptor portion (in the ‘inverse’ mode), as outlined in more detail in Example 1. In both assay configurations, test compound activity was assessed with increasing concentrations of test compounds (dose-response studies) to monitor the ability to promote CRBN-ligand-induced protein interaction—i.e., interaction with the indicated neosubstrates: IKZF1, IKZF3, IKZF2, SALL4, ZFP91 or CSNK1A (CK1a). As shown, whereas in the case of IKZF1 compound-induced binding to CRBN can be detected with both the forward and the inverse assay configuration, in all of the other cases, however, only in the inverse assay setup an interaction signal response could be detected.

FIG. 5 Immunofluorescence staining indicates that the IKZF3 gp130 fusion construct is located in the nucleus, explaining the lack of signal in the corresponding MAPPIT forward assay. As discussed in more detail in Example 1, one of the causes underlying a MAPPIT assay not being able to detect certain interactions can be that the gp130 domain fusion with the target protein of interest used in the assay is not expressed in the cytoplasmic cellular compartment and as such is unable to form a complex with the membrane-tethered bait protein. One case illustrated in FIGS. 4A-F where the compound-induced recruitment with CRBN could only be detected in the inverse mode and not in the forward assay configuration is the interaction with IKZF3. In the latter, forward assay configuration, a MAPPIT chimeric receptor fusion of CRBN is used in combination with a gp130-IKZF3 fusion protein. Here we performed an immunofluorescence staining of the gp130-IKZF3 fusion protein. A gp130-IKZF1 fusion protein was taken along as a control, as the CRBN-IKZF1 interaction was detectable in the forward mode. In the immunofluorescence images the green staining representing the gp130 fusion proteins can be observed in the cytoplasm for IKZF1, whereas in the case of the IKZF3 fusion the expression was restricted almost exclusively to the nuclear compartment (which is visualized as the blue staining). This explains why CRBN-IKZF3 interactions cannot be assayed in the forward MAPPIT configuration, but only in the inverse mode, where IKZF3 is anchored to the MAPPIT membrane receptor, with IKZF3 being exposed in the cytoplasm and available for interaction with the CRBN bait protein present in the cytoplasm.

FIG. 6 MAPPIT binding analysis indicates that the CSNK1A1-130 fusion protein exhibits strong non-specific binding to the MAPPIT chimeric receptor construct, resulting in high background reporter signal in the CRBN interaction assay in absence of a molecular glue, explaining the absence of a differential compound/molecular glue-induced response for that interaction in the forward assay configuration. As mentioned in Example 1, another reason why MAPPIT in the forward configuration might be unable to detect compound-induced interactions with particular targets is because these targets, when cloned and expressed as gp130 fusion proteins, exhibit a strong affinity for a component of the MAPPIT chimeric receptor that is not the bait protein, such as the leptin receptor cytoplasmic tail or the receptor-associated JAK2 protein. One such case, shown in FIGS. 4A-F, is CSNK1A1 (or CK1a), which clearly only exhibits a lenalidomide- or CC-220-induced and specific signal in the inverse and not in the forward MAPPIT setup. Here we performed a MAPPIT binding analysis where we tested CSNK1A1-gp130 fusion proteins (with CSNK1A1 either N- or C-terminally fused to the gp130 subdomain used in MAPPIT) for binding with MAPPIT chimeric receptor fusions with CRBN bait or a fusion with the unrelated E. coli DHFR (dihydrofolate reductase) protein. The luciferase reporter signal, which is representative for the interaction strength, indicates that the CSNK1A1-gp130 fusion interacts with/binds to both receptor fusions, suggesting that this binding is not CRBN-specific but rather that CSNK1A1 interacts with a component of the chimeric receptor itself. In the case of this configuration being used to evaluate CRBN binding proteins, the resulting high reporter signal in the absence of a specific compound-induced interaction with CRBN masks detection of any additional signal that would be induced by interaction of a particular prey-gp130 fusion with CRBN, making the forward assay setup not suitable for CRBN interaction analysis of proteins with similar behavior to that observed with CSNK1A1.

FIG. 7 Compound-dependent CRBN-CSNK1A1 interaction analysis in MAPPIT inverse configuration applying an alternative CSNK1A1 chimeric receptor fusion protein. As discussed in Example 1, multiple receptor fusion configurations can be applied in the MAPPIT assay. A typical fusion protein consists of the extracellular domain of the EPO receptor fused to the transmembrane and intracellular portion of the mutated leptin receptor, which is the construct used in FIGS. 4A-F, 6 and 8. However, the extracellular EPO receptor domain can be exchanged for that of the leptin receptor, resulting in an assay system that is activated by leptin rather than EPO. Here we recapitulated the lenalidomide- and CC-220-induced CRBN-CSNK1A1 interaction illustrated in FIGS. 4A-F using the CRBN gp130 fusion in combination with an alternative CSNK1A1 receptor fusion where the extracellular domain of the leptin receptor was used instead of that of the EPO receptor, and the assay was activated with leptin. The results show that also with this alternative receptor construct CSNK1A1 neosubstrate recruitment can be detected in the inverse assay configuration. In each set of histograms, the leftmost bar is 0 μM, the next bar to the right is 0.1 μM, the next bar to the right is 1 μM, and the rightmost bar is 10 μM.

FIGS. 8A-C Evaluation of compound-dependent FKBP1A (FKBP12)-target interactions in MAPPIT forward and inverse assay configuration. Similar to the analysis in FIGS. 4A-F for CRBN target interactions, herfie we evaluated compound-dependent FKBP1A (FKBP12) interactions with known target proteins in MAPPIT forward and inverse configurations. As shown, rapamycin-induced recruitment of MTOR is detected in both MAPPIT forward and inverse modes. Similarly, also the FK506-dependent binding of the calcineurin catalytic PPP3CA subunit can be monitored in both assay modes. Of note, in the case of calcineurin binding, co-expression of the PPP3R2 subunit increases the signal window in both assay configurations, although to a lesser extent in the inverse compared to the forward mode.

FIG. 9 Trimethoprim-lenalidomide hybrid ligand-induced binding between CRBN and DHFR can be detected in MAPPIT inverse configuration. A hybrid molecule consisting of the DHFR ligand trimethoprim (TMP) fused to the CRBN ligand lenalidomide through a PEG linker was used to induce DHFR recruitment to CRBN bait in the MAPPIT assay. As shown, in the inverse assay configuration, with CRBN expressed as a gp130 fusion and DHFR linked to the MAPPIT chimeric membrane receptor, a TMP-LEN dose-dependent signal can be observed.

FIGS. 10A-B Application of the CRBN inverse MAPPIT assay to assess CRBN binding of IMiDs and other molecular glues. Here we used the inverse MAPPIT TMP-LEN-dependent DHFR-CRBN binding assay described in FIG. 9 to evaluate binding of CRBN molecular glues in a competition setup. Cells transfected with the appropriate cDNAs encoding transgenes (DHFR and CRBN fusion proteins) were used to generate a positive assay signal by adding TMP-LEN hybrid ligand as in FIG. 9. That signal is set to 100% luciferase activity. In a separate sample set up, cells were prepared in the same manner but, in addition, co-incubated with a test compound whose interaction with CRBN is investigated. Binding to the CRBN fusion protein would compete with binding of the hybrid ligand to the same CRBN protein, hence inhibiting the assay signal due to prevention of ternary complex formation, which is required to generate an assay signal. Increasing concentrations of test compound were assessed to determine CRBN binding efficiency as determined in this type of ligand competition experiment in living cells. As shown, the known IMiD compounds (lenalidomide/LEN, pomalidomide/POM, CC-122, CC-220) competed efficiently with the lenalidomide hybrid ligand for binding to CRBN (dose-response curves for CRBN-associated assay signal inhibition). Similarly, a set of other compounds compete efficiently at varying levels of potency. Specificity of signal inhibition is assessed by a parallel experimental set up in which test compound effect is assessed for inhibition of signal generated by a control gp-130 fusion protein (CTRL) that directly binds to the DHFR receptor fusion protein in the absence of hybrid ligand (i.e. a direct interaction of the proteins).

FIG. 11 Detection of TMP-FK506-induced binding between FKBP1A (FKBP12) and DHFR using an inverse MAPPIT assay configuration. A hybrid molecule consisting of the DHFR ligand trimethoprim (TMP) fused to the FKBP1A (FKBP12) ligand FK506 through a PEG linker was used to test compound-induced binding of DHFR to FKBP1A bait in an inverse MAPPIT assay setup with FKBP1A as a gp130 fusion and DHFR linked to the chimeric membrane receptor. As shown, a clear dose-dependent MAPPIT signal can be observed, indicating that this inverse MAPPIT assay configuration is able to evaluate FK506 hybrid ligand-induced interaction between FKBP1A and DHFR.

FIG. 12 Sulfonamide-induced recruitment of RBM39 to DCAF15 can be detected in MAPPIT inverse but not in forward configuration. Similar to glue-induced substrate recruitment to CRBN, also compound-induced substrate binding for other E3 ligases was reported, for example the sulfonamide-dependent recruitment of RBM39 to DCAF15. MAPPIT was applied to assess sulfonamide-induced recruitment of RBM39 to DCAF15 in forward configuration (DCAF15 receptor fusion co-expressed with RBM39 gp130 fusion) or in inverse setup (RBM39 receptor fusion combined with DCAF15 gp130 fusion). Different sulfonamides were evaluated: indisulam (forward and inverse mode) and tasisulam, chloroquinoxaline sulfonamide (CQS) and E7820 (inverse mode). As shown, only in inverse mode could a dose-dependent luciferase signal increase be observed.

FIGS. 13A-C Screening of a compound collection identifies novel molecular glues that enable recruitment of SALL4 to CRBN. An inverse MAPPIT assay, where a CRBN gp130 fusion construct and a SALL4 chimeric receptor fusion construct were co-expressed, was used to screen a collection of 96 IMiDs and IMiD-like compounds. In a primary screen, the compounds were tested at 3 doses (low, medium and high concentration) and luciferase reporter signal was determined. The curves shown in FIGS. 13A-C represent luciferase signal frequency distributions for both compound-treated samples and DMSO-treated controls (left panel). The curve for the compound-treated samples is bimodal, where the right-shifted peak covers compounds that exhibit a reporter signal that is higher than that for the DMSO-treated controls. For three compounds exhibiting a response and thus representing compounds that induce recruitment of SALL4 to CRBN, the dose-response hit confirmation is shown (right panel). The corresponding signal at each of the tested concentrations in the primary screen is indicated by line marks with a dash type corresponding to the one used in the dose-response curves (dotted, dashed or solid). These sample curves indicate that the MAPPIT inverse approach enables the identification of molecular glues across a broad potency range.

FIG. 14 ORF cDNA library screening to identify novel molecular glue-induced CRBN neosubstrates. Here, the MAPPIT inverse approach was applied in a cell microarray-based screening format to screen a human ORF(eome) cDNA library for targets recruited to CRBN in response to CC-220, a known IMiD drug and CRBN ligand. Protein and small molecule interactions in cells were assayed within cell clusters displayed in an array format. Each spot in a cell microarray corresponded to such a cell cluster expressing a single ORF/protein candidate that is being tested for ligand-induced (in this case CC-220-induced) interaction with CRBN. A positive interaction was read out as an increase in cell fluorescence. Shown is a dot plot of the fluorescence intensity data from a cell microarray screen across/for a large number of individual ORFs/target protein candidates. The X-axis shows the Particle Count and the Y-Axis shows the integral intensity for each cell cluster in the microarray. As shown, and indicated, a significant induction of signal is observed for a number of ORF cDNAs. For three ORF cDNAs exhibiting a response and therefore representing proteins being recruited to CRBN through the CC-220 molecular glue (indicated by arrows), dose-response curves were generated to confirm their CC-220 dose-dependent binding to CRBN. These examples show that this inverse MAPPIT screening approach enables identifying novel molecular glue-induced substrates of CRBN.

FIGS. 15A-B Identification of rapamycin-induced binding between FKBP proteins and MTOR in MAPPIT forward and inverse configuration. Different members of the FKBP protein family (FKBP1A/FKBP12, FKBP3, FKBP4 and FKBP5) were evaluated in a MAPPIT assay for recruitment of MTOR (FRB domain) in either forward (FKBP receptor fusion and MTOR gp130 fusion) or inverse (MTOR receptor fusion and FKBP gp130 fusion) assay configuration. As shown, for each of the tested FKBP proteins and in both forward and inverse setup, a rapamycin-dependent signal was obtained, in line with published reports. In each set of histograms, the leftmost bar is 0 nM rapamycin, the next bar to the right is 1 nM rapamycin, the next bar to the right is 10 nM rapamycin, and the rightmost bar is 100 nM rapamycin.

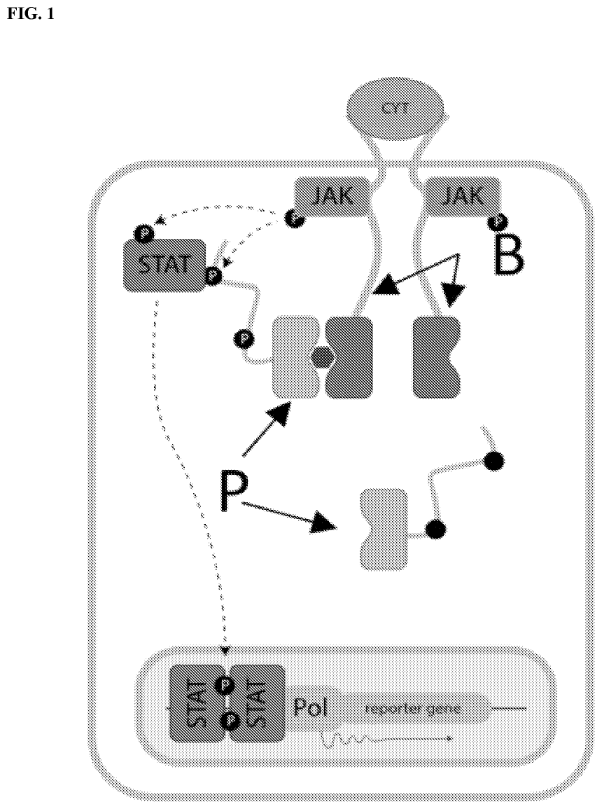

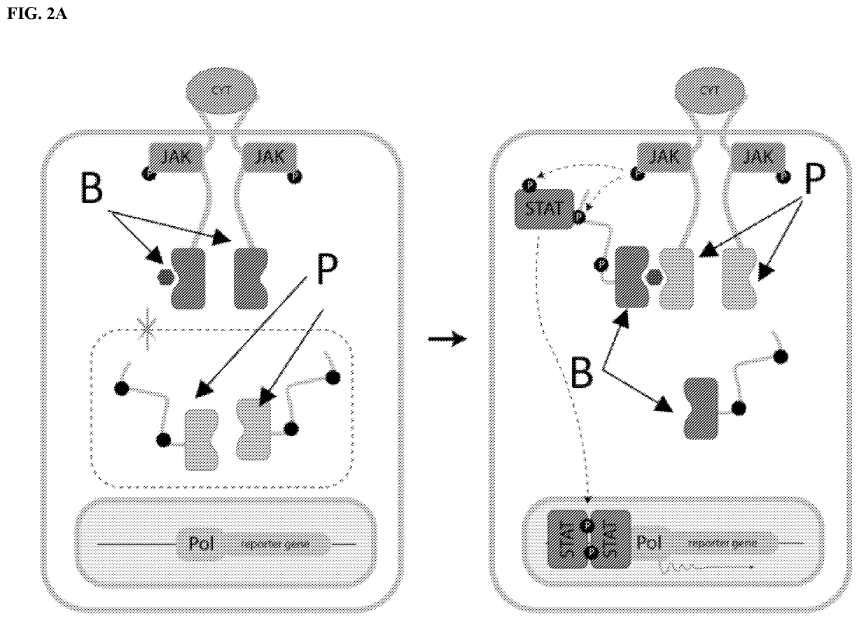

### DETAILED DESCRIPTION

The present disclosure is based, in part, on the discovery of cell-based systems and methods that allow interrogation of molecular interactions (e.g., protein/protein, protein/small molecule, and/or protein/protein interactions that are modulated by small molecules) which are not detectable using standard assays. In one aspect, the present methods allow for a method for detecting a molecular interaction, comprising: (a) providing a cell comprising a ligand-based chimeric receptor comprising (i) an extracellular portion of a ligand-binding domain derived from a first receptor and (ii) transmembrane and cytoplasmic domains of a second receptor and having an intracellular prey protein fused thereto, wherein the transmembrane and/or cytoplasmic domains of the second receptor comprise mutations that reduce or eliminate STAT recruitment; (b) expressing a bait protein that is fused to a receptor fragment in the cell, the receptor fragment comprising functional STAT recruitment sites; and (c) detecting a signal that is indicative of a molecular interaction, wherein the bait protein (i) favors occupying the cytosol over the interior of a membrane bound organelle and/or (ii) favors specific interaction with the prey protein over non-specific interaction with the cell membrane and/or a non-prey portion of the chimeric receptor.

In some embodiments, the interaction between the prey protein and bait protein causes recruitment of the receptor fragment to the cytoplasmic domain of the second receptor that is fused to a first receptor, which restores ligand-dependent receptor signaling and activation of STAT molecules. In some embodiments, the cell comprises a STAT-responsive reporter gene. In some embodiments, and the activated STAT molecules migrate to the nucleus and induce transcription of a STAT-responsive reporter gene and, in some instances, the reporter gene signal permits detection of a molecular interaction.

In some instances, a non-specific interaction of the bait protein with any portion of the ligand-based chimeric receptor, except, the prey protein, can lead to false signals or background noise in the signal obtained from the cell-based system described herein. Accordingly, in some embodiments, the bait protein specifically interacts with the prey protein or is involved in a prey protein mediated interaction. In some embodiments, the bait protein is such that it favors interactions with the prey protein over interactions with any other part of the ligand-dependent chimeric receptor. In other embodiments, the bait protein favors interactions with the prey protein over interactions with any portion of the cell membrane or other cell components. In some embodiments, the bait protein has a higher binding affinity for the prey protein than any non-prey portion of the chimeric receptor. In some embodiments, the bait protein has higher binding affinity for the prey protein as compared to any portion of the cell membrane.

Sequestration or entrapment of the bait protein within any portion of the cell such that the bait protein cannot interact with the prey protein can lead to false negative signals from the cell-based systems described herein. Accordingly, in some embodiments, the bait protein is freely available, within the cell, to interact with the prey protein. In some embodiments, the bait protein is not substantially entrapped within a cell organelle, such as, nucleus, mitochondria, Golgi apparatus, or the endoplasmic reticulum of the cell. In some embodiments, the bait protein is available within the cytosol of the cell to interact with the prey protein. In some embodiments, the bait protein does not substantially interact with the cellular membrane. In some embodiments, the bait protein does not substantially interact with the non-prey portion of the chimeric receptor. In some embodiments, the bait protein does not substantially interact with the transmembrane and/or cytoplasmic domains of the second receptor of the chimeric receptor.

In various embodiments, the present bait protein is not fused to the ligand-dependent chimeric receptor.

In various embodiments, the present prey (which in the Inverse mode is attached to a membrane protein), when assayed in forward MAPPIT (that is, not attached to a membrane protein), is limited by being trapped in the interior of a membrane bound organelle and/or non-specifically interacting with the cell membrane and/or a non-prey portion of the chimeric receptor. Further, in forward MAPPIT, the bait may express poorly as a receptor fusion protein and/or not fold properly and/or the fusion may obscure an interaction face of the bait and therefore, in embodiments, in the inverse MAPPIT, making the bait soluble (i.e. not fused) solves these issues.

In various embodiments, the prey protein is one that is undetected or poorly detected when analyzed as a gp130 fusion in forward MAPPIT. In various embodiments, a molecular interaction, e.g., without limitation, a protein/protein interaction or a protein/protein interaction which is mediated by the binding of a small molecule with the prey protein or bait protein, is detected in the present methods and is undetected or poorly detected when analyzed in forward MAPPIT.

In various embodiments, the bait protein is not fused to a transmembrane protein and therefore may expose patches of protein structure that are more analogous to what is intrinsic to physiological conditions. Stated another way, the lack of bait fusion may more accurately reflect the condition of the bait in natural situations.

The present invention also includes analyzing a library of prey proteins, a library of ligand-based chimeric receptors, and/or a library of cells expressing the library of ligand-based chimeric receptors for molecular interactions. The present invention also includes analyzing a library of compounds. In embodiments, the bait binds to the compound and, optionally this bait-compound complex interacts with the prey. In embodiments, therefore, the present methods allow for the detection and/or discovery of novel compound mediated protein/protein interactions and/or novel protein/compound interactions. In embodiments, the present methods allow for the detection and/or discovery of novel compounds that act as molecular glues. In embodiments, the present methods allow for the detection and/or discovery of novel compound which converts a weak bait-prey interaction into a stronger bait-prey interaction.

For example, the prey library includes at least two different types/kinds of unique prey proteins that are suitable for carrying out methods described herein.

The receptor library of the present invention includes at least two ligand-based chimeric receptors where each chimeric receptor or every population of chimeric receptors is fused to a different type/kind of prey protein. The cell library includes at least two different kinds/types of cells where each cell or a population of cells expresses one type/kind of chimeric protein such that the chimeric protein is fused to one or more kinds/types of prey protein.

In some embodiments, the cell library includes a first population/fraction of cells where the first population/fraction of chimeric receptors is fused to a first kind of prey protein and a second population/fraction of cells where the second population/fraction of chimeric receptors is fused to a second kind of prey protein. The number of populations of cells included in the cell library is not limited. For instance, in one embodiment, the cell library includes two or more different cell populations where each population of cells is different from the other population because it has a different prey protein fused to the chimeric receptor.

In some embodiments, the receptor library includes a first population of chimeric receptors where the chimeric receptors are fused to one kind/type of prey protein and a second population of chimeric receptors where the receptors are fused to another kind/type of prey protein. There is no limitation on the number of different types/kinds of chimeric receptors that may be included in the receptor library. For instance, in one embodiment, the receptor library includes two or more different chimeric receptor populations where each population of receptors is different from the other population because it has a different prey protein fused to the chimeric receptors.

In various embodiments, the present methods pertain to an open reading frame (ORF) library of prey proteins fused to chimeric receptors. In various embodiments, the present methods pertain to a population of cells having an ORF library of prey proteins fused to chimeric receptors. In embodiments, such ORF libraries, in the context of prey proteins fused to chimeric receptors, can be used to interrogate a single bait, which is not fused to chimeric receptors.

In various embodiments, the present methods pertain to an array-based format, e.g. in which cDNAs encoding various bait proteins are spotted on a surface. In various embodiments. the present methods pertain to a cell population-based method in which, e.g. a library of bait proteins is introduced into cells such that, on average, each cell expresses a single bait. In such embodiments, upon interaction with compound and/or bait, the encoding cDNA is identified to reveal the interactions. In embodiments, FACS or microfluidic separation is employed for the identification.

In various embodiments, a plurality of prey proteins are analyzed for molecular interaction with a single bait, the bait not being fused to the ligand-dependent chimeric receptor.

In some embodiments, the molecular interaction is a protein/protein interaction. In embodiments, the bait and prey are both proteins.

In some embodiments, the method further comprises introducing a small molecule which binds to the prey protein or bait protein. In some embodiments, the molecular interaction is a protein/protein interaction which is mediated by the binding of the small molecule with the prey protein or bait protein. In embodiments, the bait and prey are both proteins.

In some embodiments, the molecular interaction is two or more protein/protein interactions which are mediated by the binding of the small molecule with the prey protein or bait protein. In some embodiments, the protein/protein interaction which is mediated by the binding of the small molecule with the prey protein or bait protein is a direct binding between the prey protein or bait protein and the small molecule at a protein/protein interface.

In some embodiments, the protein/protein interaction which is mediated by the binding of the small molecule with the prey protein or bait protein is mediated by an allosteric modification of the protein surface of the prey protein or bait protein.

In some embodiments, the small molecule induces exposure of a hydrophobic surface of the prey protein or bait protein that allows for interaction with the prey protein or bait protein. In some embodiments, the small molecule induces exposure of a hydrophobic surface of the bait protein that allows for interaction with the prey protein. In some embodiments, the small molecule induces exposure of a hydrophobic surface of the prey protein that allows for interaction with the bait protein.

In some embodiments, the small molecule is a molecular glue. In some embodiments, the small molecule is a bivalent hybrid ligand molecule (e.g., without limitation, a PROTAC).

In some embodiments, the molecular interaction is a complex formation.

In some embodiments, the molecular interaction is a small molecule/protein interaction.

In some embodiments, the prey protein is bound to a small molecule and the small molecule is connected via a linker to a second small molecule which binds to the bait protein. In some embodiments, the bait protein is bound to a small molecule and the small molecule is connected via a linker to a second small molecule which binds to the prey protein.

In various embodiments, the first receptor and second receptor are the same.

In various embodiments, the first receptor and second receptor are different.

In various embodiments, the first receptor and/or second receptor is a multimerizing receptor.

In some embodiments, the ligand-binding domain is derived from a cytokine receptor. In some embodiments, the ligand-binding domain is derived from a Type 1 cytokine receptor (CR).

In some embodiments, the ligand-binding domain is derived from erythropoietin receptor (EpoR) or leptin receptor. In some embodiments, the transmembrane and cytoplasmic domains are derived from the murine leptin receptor.

In some embodiments, the bait is heterologous to the first receptor and/or second receptor fragment.

In some embodiments, the cytoplasmic domain comprises a JAK binding site.

In some embodiments, the cytoplasmic domain comprises glycoprotein 130 (gp130) or a fragment thereof.

In some embodiments, the receptor fragment comprises glycoprotein 130 (gp130) or a fragment thereof.

In some embodiments, the STAT is selected from STAT1 or STAT3.

In some embodiments, the mutations that reduce or eliminate STAT recruitment are to one or more tyrosine phosphorylation sites. In some embodiments, the transmembrane and cytoplasmic domains are derived from the murine leptin receptor and the mutations are at one or more of positions Y985, Y1077, and Y1138. In some embodiments, the transmembrane and cytoplasmic domains are derived from the murine leptin receptor and the mutations are Y985F, Y1077F, and Y1138F. In some embodiments, the transmembrane and cytoplasmic domains have functionally equivalent mutations to Y985F, Y1077F, and Y1138F of the murine leptin receptor. In some embodiments, there is provided a deletion of a transmembrane domain, provided that JAK binding is retained.

The amino acid sequence of the murine leptin receptor is as follows:

(SEQ ID NO: 1)
MMCQKFYVVLLHWEFLYVIAALNLAYPISPWKFKLFCGPPNTTDDSFLSP
AGAPNNASALKGASEAIVEAKFNSSGIYVPELSKTVFHCCFGNEQGQNCS
ALTDNTEGKTLASVVKASVFRQLGVNWDIECWMKGDLTLFICHMEPLPKN
PFKNYDSKVHLLYDLPEVIDDSPLPPLKDSFQTVQCNCSLRGCECHVPVP
RAKLNYALLMYLEITSAGVSFQSPLMSLQPMLVVKPDPPLGLHMEVTDDG
NLKISWDSQTMAPFPLQYQVKYLENSTIVREAAEIVSATSLLVDSVLPGS
SYEVQVRSKRLDGSGVWSDWSSPQVFTTQDVVYFPPKILTSVGSNASFHC
IYKNENQIISSKQIVWWRNLAEKIPEIQYSIVSDRVSKVTFSNLKATRPR
GKFTYDAVYCCNEQACHHRYAELYVIDVNINISCETDGYLTKMTCRWSPS
TIQSLVGSTVQLRYHRRSLYCPDSPSIHPTSEPKNCVLQRDGFYECVFQP
IFLLSGYTMWIRINHSLGSLDSPPTCVLPDSVVKPLPPSNVKAEITVNTG
LLKVSWEKPVFPENNLQFQIRYGLSGKEIQWKTHEVFDAKSKSASLLVSD
LCAVYVVQVRCRRLDGLGYWSNWSSPAYTLVMDVKVPMRGPEFWRKMDGD
VTKKERNVTLLWKPLTKNDSLCSVRRYVVKHRTAHNGTWSEDVGNRTNLT
FLWTEPAHTVTVLAVNSLGASLVNFNLTFSWPMSKVSAVESLSAYPLSSS
CVILSWTLSPDDYSLLYLVIEWKILNEDDGMKWLRIPSNVKKFYIHDNFI
PIEKYQFSLYPVFMEGVGKPKIINGFTKDAIDKQQNDAGLYVIVPIIISS
CVLLLGTLLISHQRMKKLFWDDVPNPKNCSWAQGLNFQKPETFEHLFTKH
AESVIFGPLLLEPEPISEEISVDTAWKNKDEMVPAAMVSLLLTTPDPESS
SICISDQCNSANFSGSQSTQVTCEDECQRQPSVKYATLVSNDKLVETDEE
QGFIHSPVSNCISSNHSPLRQSFSSSSWETEAQTFFLLSDQQPTMISPQL
SFSGLDELLELEGSFPEENHREKSVCYLGVTSVNRRESGVLLTGEAGILC
TFPAQCLFSDIRILQERCSHFVENNLSLGTSGENFVPYMPQFQTCSTHSH
KIMENKMCDLTV.

In some embodiments, the domains are derived from the murine leptin receptor are amino acids 839-1162 of the murine leptin receptor sequence.

In some embodiments, the bait protein comprises a nuclear export sequence (NES). For example, in embodiments, the bait protein is a nuclear protein and the NES ensures that it is available in the cytosol (i.e. to contact the prey, if applicable). Thus, in embodiments, the NES signal helps keep the bait polypeptide in the cytoplasm even when a strong nuclear localization signal is present, thus facilitating interaction with the prey.

In some embodiments, the NES has 1-4 hydrophobic residues. In some embodiments, the hydrophobic residues are leucines. In some embodiments, the NES has the sequence LxxxLxxLxL, where L is a hydrophobic residue and x is any other amino acid. In some embodiments, the NES has the sequence LxxxLxxLxL, where L is a leucine and x is any other amino acid.

In some embodiments, the NES comprises amino acids 37-46 of the heat-stable inhibitor of the cAMP-dependent protein kinase, which has been shown to override a strong nuclear localization signal (Wiley et al., (1999), J. Biol. Chem. 274:6381-6387, the entire contents of which are incorporated by reference).

In some embodiments, the present methods allow for the identification of new interaction partners, e.g., substrates or neosubstrates of a protein that binds to a compound, the protein having a cage of three tryptophan residues that are capable of interacting with a glutarimide ring of the compound, e.g., via hydrogen binding. In some embodiments, the interaction partner, e.g., neosubstrate, has a surface β-hairpin loop, the surface β-hairpin loop optionally having an arrangement of three backbone hydrogen bond acceptors at the apex of a turn followed by a glycine residue. In some embodiments, the interaction partner, e.g., neosubstrate, has a degron motif (see, Meszaros, et al. Sci Signal 2017: 10, 470, the entire contents of which are incorporated by reference).

In some embodiments, the bait is a protein having a cage of three tryptophan residues that are capable of interacting with a glutarimide ring of the compound (such as, immunomodulatory drugs or immunomodulatory imide drugs (IMiDs)), e.g., via hydrogen binding.

In some embodiments, the prey, e.g., neosubstrate, has a surface β-hairpin loop, the surface β-hairpin loop optionally having an arrangement of three backbone hydrogen bond acceptors at the apex of a turn followed by a glycine residue. In some embodiments, the prey, e.g., neosubstrate, has a degron motif (see, Meszaros, et al. Sci Signal 2017: 10, 470, the entire contents of which are incorporated by reference).

In some embodiments, the bait is a protein that modulates the ubiquitin-proteasome system. In some embodiments, the bait is an E3 ligase protein, or a protein that modulates an E3 ligase protein. In some embodiments, the bait is a cullin-RING ligase (CRL) protein, or a protein that modulates an CRL protein. In various embodiments, the bait is a CRL4 protein, or a protein that modulates an CRL4 protein. In some embodiments, the bait is a DDB1-CUL4-associated factor (DCAF) protein, or a protein that modulates a DCAF.

In some embodiments, the bait is or comprises one or more of cereblon (CRBN), damaged DNA binding protein 1 (DDB1), Cullin-4A (CUL4A), regulator of cullins 1 (ROC1), and Von Hippel Lindau (VHL). In various embodiments, the bait is one or more of cereblon (CRBN), damaged DNA binding protein 1 (DDB1), Cullin-4A (CUL4A), regulator of cullins 1 (ROC1), and Von Hippel Lindau (VHL). In various embodiments, the prey is one or more of cereblon (CRBN), damaged DNA binding protein 1 (DDB1), Cullin-4A (CUL4A), regulator of cullins 1 (ROC1), and Von Hippel Lindau (VHL).

In some embodiments, the bait is or comprises an FK506 binding protein (FKBP), optionally selected from FKBP12, FKBP38 and FKBP52.

In some embodiments, the bait protein is fused to a receptor fragment. In some embodiments, the bait protein is fused to a receptor fragment, either N- or C-terminally. In some embodiments, the bait protein is fused to gp130 or a fragment thereof. In some embodiments, the bait protein is fused to gp130 or a fragment thereof, either N- or C-terminally.

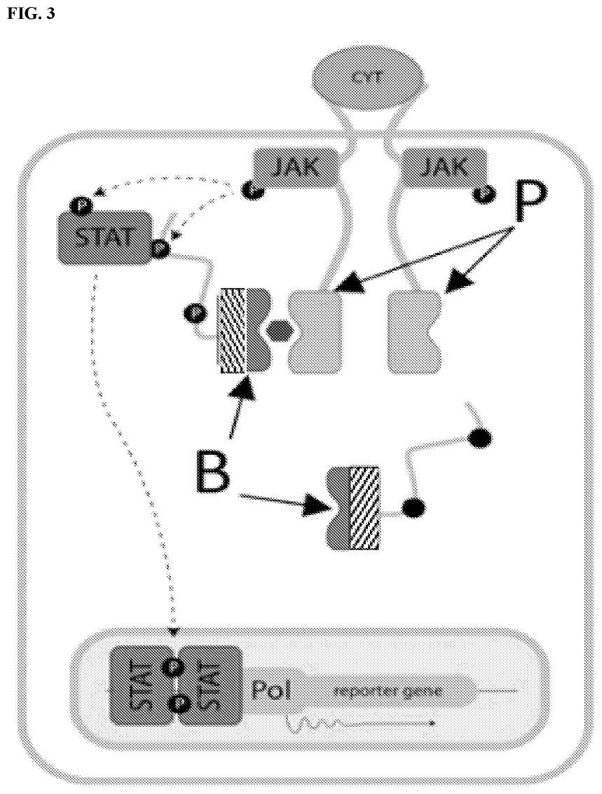

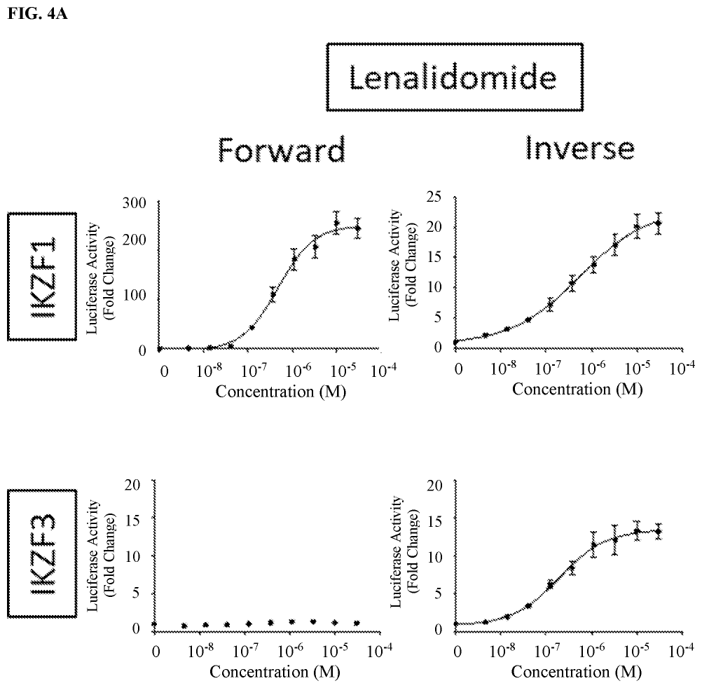

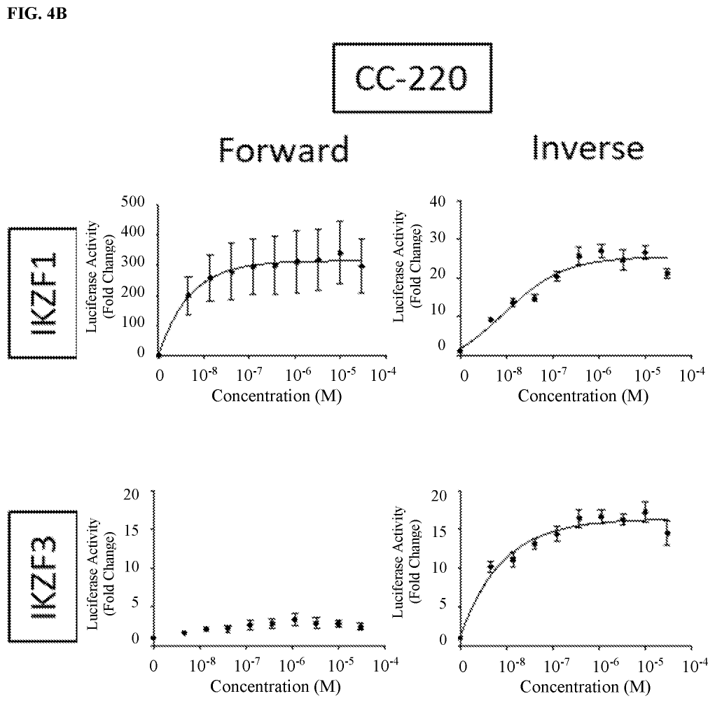

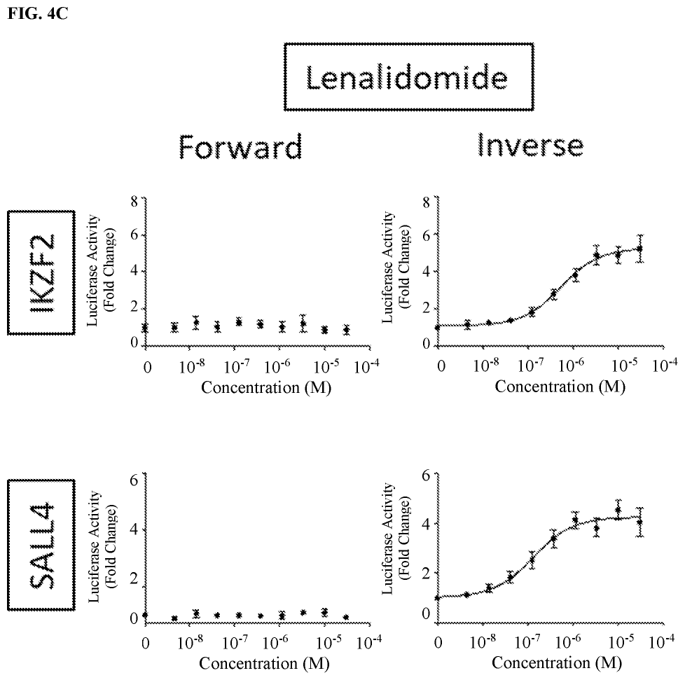

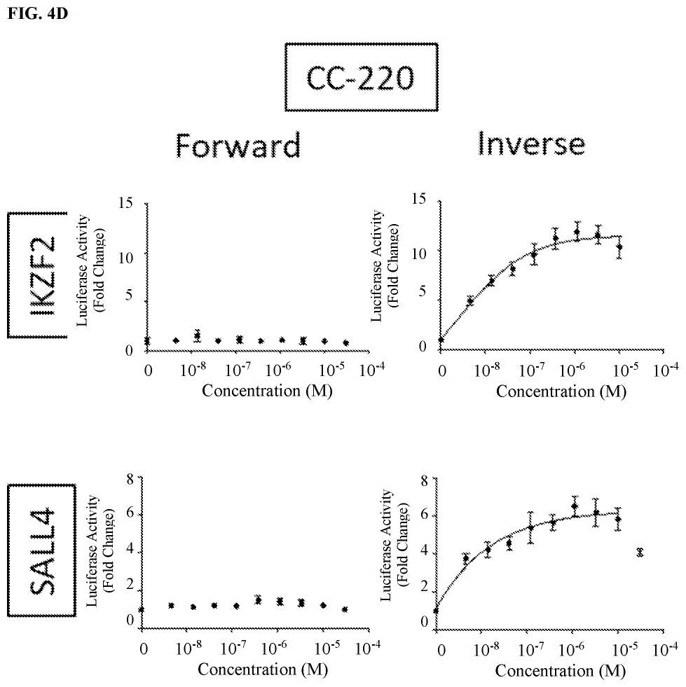

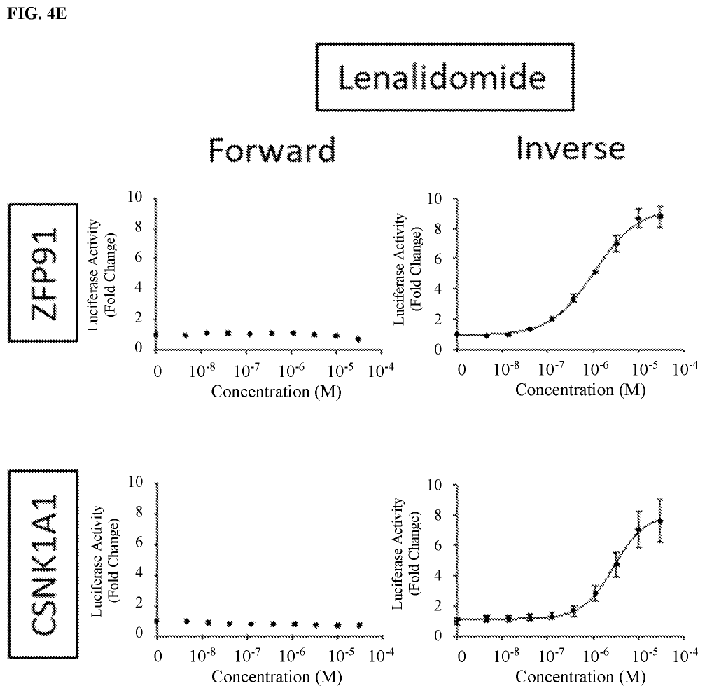

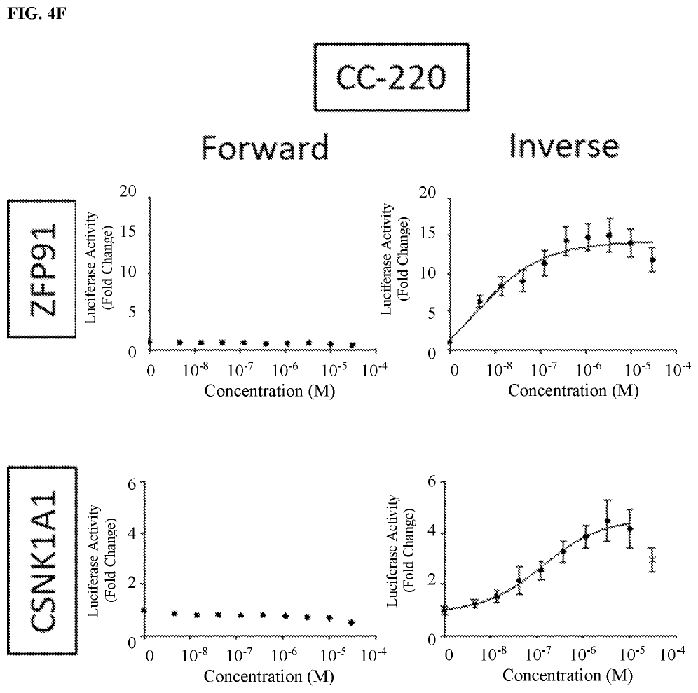

In embodiments, the bait is more than one protein (e.g. 2, or 3, or 4, or 5 proteins). In embodiments, the bait comprises a first protein that can interact with the small molecule and/or bait and a scaffold protein that interacts with the first protein. In some embodiments, the present methods employ the system of FIG. 3.

In some embodiments, the scaffold protein is fused to a receptor fragment. In some embodiments, the scaffold protein is fused to a receptor fragment, either N- or C-terminally. In embodiments, the scaffold protein is fused to gp130 or a fragment thereof. In embodiments, the scaffold protein is fused to gp130 or a fragment thereof either N- or C-terminally.

In some embodiments, the scaffold protein is fused to a receptor fragment. In embodiments, the scaffold protein is fused to gp130 or a fragment thereof and the protein that can interact with the small molecule and/or bait interacts with the scaffold protein and can interact with the prey and/or a small molecule.

In embodiments, the bait is an E3 ligase substrate binding subunit.

In embodiments, the E3 ligase substrate binding subunit is selected from the protein encoded by any of the following genes: AMFR, ANAPC11, APG16L, ARIH1, ARIH2, ARPC1A, ARPC1B, ASB2, ASB2, ATG16L1, BAF250, BARD1, BIRC2, BIRC3, BIRC4, BIRC7, BMI1, BRAP, BRCA1, bTrCP, CBL, CBLB, CBLC, CBLL1, CCIN, CCIN, CCNB1IP1, CRBN, CHFR, CHIP, CNOT4, COP1, CSA, DCAF1, DCAF10, DCAF11, DCAF12, DCAF13, DCAF14, DCAF15, DCAF16, DCAF17, DCAF19, DCAF2, DCAF3, DCAF4, DCAF5, DCAF6, DCAF7, DCAF8, DCAF9, Dda1, DDB2, DET1, DNAI2, DTX3, DZIP3, E6AP, EDD, EED, ENC1, ENC1, FANCL, FBXL1, FBXL10, FBXL11, FBXL12, FBXL13, FBXL14, FBXL15, FBXL16, FBXL17, FBXL18, FBXL19, FBXL20, FBXL21, FBXL22, FBXL3, FBXL4, FBXL5, FBXL7, FBXL8, FBXO1, FBXO10, FBXO11, FBXO12, FBXO13, FBXO14, FBXO15, FBXO16, FBXO17, FBXO18, FBXO19, FBXO2, FBXO20, FBXO21, FBXO22, FBXO3, FBXO4, FBXO5, FBXO6, FBXO7, FBXO8, FBXW1, FBXW10, FBXW11, FBXW12, FBXW5, FBXW7, FBXW8, FBXW9, FEM1A, FEM1B, FEM1C, GAN, GAN, GNB1, GNB2, GNB5, GRWD1, GTF2H2, GTF3C2, HACE1, HECTD1, HECTD2, HECTD3, HERC1, HERC2, HERC3, HERC4, HERC5, HERC6, HLTF, HOIP, HUWE1, IBRDC2, IBRDC3, IFRG15, IPP, IPP, ITCH, IVNS1ABP, IVNS1ABP, KATNB1, KBTBD10, KBTBD10, KBTBD11, KBTBD11, KBTBD12, KBTBD12, KBTBD13, KBTBD13, KBTBD2, KBTBD2, KBTBD3, KBTBD3, KBTBD4, KBTBD4, KBTBD5, KBTBD5, KBTBD6, KBTBD6, KBTBD7, KBTBD7, KBTBD8, KBTBD8, KCTD5, KEAP, KEAP1, KIAA0317, KIAA0614, KLHDC5, KLHL1, KLHL1, KLHL10, KLHL10, KLHL11, KLHL11, KLHL12, KLHL12, KLHL13, KLHL13, KLHL14, KLHL14, KLHL15, KLHL15, KLHL17, KLHL17, KLHL18, KLHL18, KLHL2, KLHL2, KLHL20, KLHL21, KLHL21, KLHL22, KLHL22, KLHL23, KLHL23, KLHL24, KLHL24, KLHL25, KLHL25, KLHL26, KLHL26, KLHL28, KLHL28, KLHL29, KLHL29, KLHL3, KLHL3, KLHL30, KLHL30, KLHL31, KLHL31, KLHL32, KLHL32, KLHL33, KLHL33, KLHL34, KLHL34, KLHL35, KLHL35, KLHL36, KLHL36, KLHL38, KLHL38, KLHL4, KLHL4, KLHL5, KLHL5, KLHL6, KLHL6, KLHL7, KLHL7, KLHL8, KLHL8, KLHL9, KLHL9, LINCR, LNX1, LRR1, LRRC41, LRSAM1, LZTR1, LZTR1, MAGEA1, MAGE-A1, MAGEA2, MAGE-A2, MAGEA3, MAGE-A3, MAGEA6, MAGE-A6, MAGEB18, MAGE-B18, MAGEB2, MAGE-B2, MAGEC2, MAGE-C2, MALIN, MAP3K1, MARCH1, MARCH11, MARCH2, MARCH4, MARCH5, MARCH6, MARCH7, MARCH8, MARCH9, MDM2, MDM4, MEX, MGRN1, MIB1, MIB2, MID1, MKRN1, MNAT1, MUF1, MULAN, MURF, MYCBP2, MYLIP, Nedd4, NEDD4L, NEDL1, NEDL2, NEURL, NEURL2, NLE1, NUP43, OSTM1, PAFAH1B1, PARC, PARK2, PCGF1, PCGF2, PDZRN3, PEX10, PEX7, PJA1, PJA2, POC1A, PPIL2, PRAME, PRPF19, PWP1, RACK1, RAD18, RAE1, RAG1, RBBP4, RBBP5, RBBP6, RBBP7, RBCK1, RBX1, RCHY1, RFFL, RFPL4A, RFWD2, RING1, RNF103, RNF11, RNF111, RNF114, RNF12, RNF123, RNF125, RNF128, RNF13, RNF130, RNF133, RNF135, RNF138, RNF139, RNF14, RNF144A, RNF167, RNF168, RNF180, RNF181, RNF182, RNF185, RNF19, RNF2, RNF20, RNF20, RNF216, RNF25, RNF34, RNF4, RNF40, RNF41, RNF43, RNF43, RNF5, RNF6, RNF7, RNF8, RNF85, RPTOR, SCAP, SH3RF1, SHPRH, SIAH1, SIAH2, SMU1, SMURF1, SMURF2, SOCS1, SOCS3, SPOP, SPSB1, SPSB1, SPSB2, SPSB2, SPSB4, SPSB4, STXBP5L, SYVN1, TAF5L, TBL1Y, THOC3, TLE1, TLE2, TLE3, TOPORS, TRAF2, TRAF6, TRAF7, TRAIP, TRIAD3, TRIM1, TRIM10, TRIM11, TRIM12, TRIM13, TRIM14, TRIM15, TRIM16, TRIM17, TRIM18, TRIM2, TRIM21, TRIM22, TRIM23, TRIM24, TRIM25, TRIM26, TRIM27, TRIM28, TRIM29, TRIM29, TRIM3, TRIM31, TRIM32, TRIM33, TRIM36, TRIM37, TRIM39, TRIM40, TRIM41, TRIM44, TRIM45, TRIM47, TRIM5, TRIM50, TRIM52, TRIM54, TRIM55, TRIM58, TRIM59, TRIM62, TRIM65, TRIM66, TRIM7, TRIM71, TRIM8, TRIM9, TRIP12, TRPC4AP, TSSC1, UBE3B, UBE3C, UBE4A, UBE4B, UBOX5, UBR1, UBR2, UBR3, UBR4, UHRF1, UHRF2, VHL, VPS18, WDR12, WDR23, WDR26, WDR3, WDR31, WDR37, WDR39, WDR4, WDR47, WDR48, WDR5, WDR51B, WDR53, WDR57, WDR59, WDR5B, WDR61, WDR76, WDR77, WDR82, WDR83, WDR86, WSB1, WSB2, WWP1, WWP2, ZNF294, ZNF313, ZNF364, ZNRF1, ZNRF2, ZYG11A, ZYG11B, or ZYG11BL.

In embodiments, the E3 ligase substrate binding subunit is CRBN or VHL.

In embodiments, the scaffold protein interacts with an E3 ligase substrate binding subunit and the complex of scaffold protein and E3 ligase substrate binding subunit interacts with the prey or its interaction is induced or mediated by a small molecule.

In embodiments, the scaffold protein is selected from BIRC6, CUL3, DDB1, ELOB, ELOC, RBX1, SKP1, UBCH5A, UBE2A, UBE2B, UBE2B2, UBE2C, UBE2D1, UBE2D2, UBE2D3, UBE2D4, UBE2E1, UBE2E2, UBE2E3, UBE2F, UBE2G1, UBE2G2, UBE2H, UBE2J1, UBE2J2, UBE2K, UBE2L3, UBE2L6, UBE2M, UBE2N, UBE2NL, UBE20, UBE2Q1, UBE2Q2, UBE2QL, UBE2R1, UBE2R2, UBE2S, UBE2T, UBE2U, UBE2V1, UBE2V1, UBE2V2, and UBE2W.

In some embodiments, the scaffold protein is selected from damaged DNA binding protein 1 (DDB1), Cullin-4A (CUL4A), and regulator of cullins 1 (ROC1).

In some embodiments, the prey is a substrate and/or neosubstrate of CRBN. In embodiments, the substrate and/or neosubstrate of CRBN comprises b-hairpin a-turn with an i-residue bearing a side chain with a hydrogen bond acceptor, such as Asx or ST motifs, with a hydrogen bond between the sidechain of i and the backbone NH of i+3 and between the backbone carbonyl oxygen of i and the backbone NH of i+4. In embodiments, the i+4 residue is glycine (non-limiting examples include GSPT1, CK1a). In embodiments, the substrate and/or neosubstrate of CRBN has a b-hairpin a-turn with residues i and i+3 being cysteine and the i+4 residue being glycine. The two Cys residues bind to a zinc ion to enforce the shape of the turn (non-limiting examples include IKZF1, ZnF692 and all the substrate reported in “Defining the human C2H2 zinc finger degrome targeted by thalidomide analogs through CRBN”, Sievers et al, Science Vol. 362, Issue 6414, DOI: 10.1126/science.aat0572 (2018), incorporated by reference in its entirety). In embodiments, the substrate and/or neosubstrate of CRBN has a “pseudo-loop”, a b-hairpin b-turn bearing a glycine in the i+3 position. Turn structure can be enforced by a hydrogen bond between a hydrogen bond acceptor of the i−1 side chain and the carbonyl of the i+3 glycine.

In some embodiments, the prey is or comprises one or more of Ikaros (IKZF1), Helios (IKZF2), Aiolos (IKZF3), Eos (IKZF4), Pegasus (IKZF5), SALL4, CSNK1A, CK1a, and ZFP91. In various embodiments, the prey is one or more of Ikaros (IKZF1), Helios (IKZF2), Aiolos (IKZF3), Eos (IKZF4), Pegasus (IKZF5), SALL4, CSNK1A, CK1a, and ZFP91. In some embodiments, the prey is one or more of Ikaros (IKZF1), Helios (IKZF2), Aiolos (IKZF3), Eos (IKZF4), Pegasus (IKZF5), SALL4, CSNK1A, CK1a, and ZFP91.

In some embodiments, the compound is an immunomodulatory agent. In some embodiments, the compound is a derivative of glutamic acid that comprises a glutarimide ring, optionally, and a phthalimide ring. In some embodiments, the phthalimide ring is chemically modified. In some embodiments, the derivative of glutamic acid can be a synthetic derivative having the properties in accordance with embodiments of the present disclosure.

In some embodiments, the compound is a member of the class of compounds known as immunomodulatory drugs or immunomodulatory imide drugs (IMiDs).

In embodiments, the compound contains a IMiD-like glutarimide ring, but otherwise differs in chemical structure and binds to the same or similar small molecule binding pocket as a glutaramide-IMiD in CRBN (the IMiD binding pocket in CRBN). In embodiments, the compound does not contain a glutaramide ring and can bind CRBN in the IMiD pocket. In embodiments, the compound binds CRBN, but not in the IMiD pocket. In embodiments, the compounds binds CRBN in a manner that is non-competitive with an IMiD or IMiD-like compound that binds in the IMiD pocket.

In some embodiments, the compound is thalidomide, lenalidomide, pomalidomide, CC-220, CC-122, CC-885, or a derivative, analog, enantiomer or a mixture of enantiomers, or a pharmaceutically acceptable salt, solvate, hydrate, co-crystal, clathrate, or polymorph thereof or a compound that binds to the same CRBN bait binding site as or a compound that binds to the same FKBP bait binding site as the FK506 (tacrolimus), rapamycin (sirolimus), and cyclosporin A (CsA) or a derivative or analog thereof and in a competitive fashion and in a competitive fashion.

In some embodiments, the compound is selected from FK506 (tacrolimus), rapamycin (sirolimus), and cyclosporin A (CsA) or a derivative or analog thereof or a compound that binds to the same FKBP bait binding site as the FK506 (tacrolimus), rapamycin (sirolimus), and cyclosporin A (CsA) or a derivative or analog thereof and in a competitive fashion.

In some embodiments, the compound is avadomide, endomide, iberdomide, lenalidomide, mitindomide, pomalidomide, and thalidomide, or a derivative, analog, enantiomer or a mixture of enantiomers, or a pharmaceutically acceptable salt, solvate, hydrate, co-crystal, clathrate, or polymorph thereof.

In some embodiments, the present method involves one or more of CRBN, DDB1, CUL4A, ROC1, and VHL as bait, and the bait is contacted with a compound described herein (e.g., a compound that binds to one or more of CRBN, DDB1, CUL4A, ROC1, and VHL, e.g., an IMiD) to discover a prey that interacts with the bait, as the bait is modulated by the compound. For example, the method identifies an interacting prey that contacts the bait, the bait being modified by the compound. In some embodiments, the prey is recruited and/or degraded because of the interaction with bait. In such embodiments, without limitation, the compound does not directly interact with the prey (e.g. by way of non-limitation, in complex with bait and prey, the compound may not contact the prey).

In some embodiments, the present methods allow for the identification of new substrates or neosubstrates of CRBN.

In various embodiments, the present methods identify a novel molecular interaction. In various embodiments, the present methods identify a novel protein/protein interaction. In various embodiments, the present methods identify a novel protein/protein interaction which is mediated by the binding of a small molecule with the prey protein or bait protein.

In various embodiments, the present methods identify a molecular interaction which is undetected or poorly detected when analyzed in forward MAPPIT. In various embodiments, the present methods identify a protein/protein interaction which is undetected or poorly detected when analyzed in forward MAPPIT. In various embodiments, the present methods identify a protein/protein interaction which is mediated by the binding of a small molecule with the prey protein or bait protein which is undetected or poorly detected when analyzed in forward MAPPIT.

In various embodiments, the present methods provide lower background signals than seen in forward MAPPIT. In various embodiments, the present methods provide less false positive signals than seen in forward MAPPIT.

In various embodiments, the present invention provides for the detection of a molecular interaction by employing both forward MAPPIT and the method described herein. Accordingly, this combined method allows for the detection of molecular interactions that are not seen when a single method, i.e. forward MAPPIT and the method described herein, is employed.

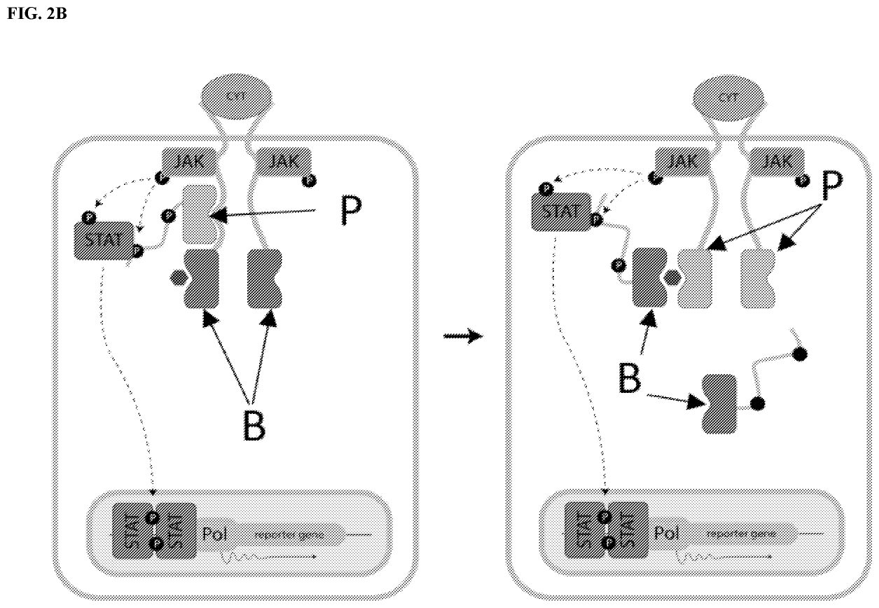

In some embodiments, the present methods employ the system of FIG. 2A (right panel).

In some embodiments, the present methods employ the system of FIG. 3.

### EXAMPLES

### Example 1: Comparison of MAPPIT Forward and Inverse Assay Configuration for the Detection of Molecular Glue-Induced CRBN Substrate Interactions

In order to identify ligand-induced CRBN substrates, or neosubstrates, here we use the MAPPIT assay, applying the procedure described in Lemmens, et al. “MAPPIT, a mammalian two-hybrid method for in-cell detection of protein-protein interactions,” Methods Mol Biol. 2015; 1278:447-55. The traditional MAPPIT assay has been used to monitor protein-protein interactions. A bait protein (protein A) is expressed as a fusion protein in which it is genetically fused to an engineered intracellular receptor domain of the leptin receptor, which is itself fused to the extracellular domain of the erythropoietin (Epo) receptor. Binding of Epo ligand to the Epo receptor component results in activation of receptor-associated intracellular JAK2. However, activated JAK2 cannot activate the leptin receptor to trigger STAT3 binding and its phosphorylation because its tyrosine residues, normally phosphorylated by activated JAK2, have been mutated. Reconstitution of a JAK2 phosphorylatable STAT3 docking site is instead created through interaction of a protein B with protein A, whereby protein B is fused to a cytoplasmic domain of the gp130 receptor (which now harbors appropriate tyrosine resides recognized by the activated JAK2 kinase). Thus, physical interaction of protein A with protein B, or formation of a protein complex that comprises protein A and protein B, reconstitutes and EPO triggered JAK2-STAT3 signaling pathway activation. Activation of STAT3 can be monitored by introduction of a STAT3-responsive reporter gene, including a luciferase-encoding gene or a gene encoding a fluorescent marker such as GFP or some other type of Fluorescent Protein (EGFP etc.). In this manner, the MAPPIT assay provides a versatile assay to assess such recombinant protein-protein interactions in intact cells.

In this Example 1, we used a derivative of the MAPPIT assay that we developed specifically for use in determining CRBN-ligand induced protein interactions, i.e. using a specific CRBN bait protein and assaying for ligand-dependent induction of protein complex formation. In the traditional MAPPIT assay configuration reported before, referred to here as ‘forward’, the bait protein of interest (CRBN in this Example 1) is expressed as a fusion with the MAPPIT chimeric membrane receptor and the interacting target protein is fused with the cytoplasmic gp130 receptor fragment (IKZF1, IKZF3, IKZF3, SALL4, ZFP91 or CSNK1A1 in the cases shown in this Example 1). Here we exemplify an alternative assay mode, termed the ‘inverse’ assay configuration, where the bait and prey fusions are inversed, i.e. the CRBN bait is fused to the gp130 fragment and the substrate proteins are fused to the transmembrane chimeric receptor.

This inverse assay configuration has a number of advantages over the classic forward mode. First, target proteins of interest which are naturally localized to other cellular compartments than the cytoplasm would not be accessible for interaction with the membrane anchored bait (here CRBN) when used as a gp130 fusion. In this case, inversing the setup where the target protein is linked to the MAPPIT chimeric transmembrane receptor and as such forced to localize in the cytoplasm, would solve that problem. One such case is IKZF3, which is discussed in Example 2.

Another instance where it is beneficial to use the target/prey protein in the MAPPIT receptor fusion rather than the gp130 fusion construct is when the target/prey protein as a gp130-fusion protein exhibits affinity for a component of the MAPPIT chimeric receptor other than the interacting protein bait, e.g. the intracellular portion of the leptin receptor or JAK2. In this case, this ‘stickiness’ results in a high reporter signal already building up in the absence of a specific bait-prey interaction, which might obscure any additional signal increase induced by a specific protein-protein or compound-induced bait-prey interaction. By reversing the configuration and having the target/prey protein fused to the MAPPIT chimeric receptor instead, any such background signal is prevented. In Example 3 this is exemplified for the compound-induced interaction between CRBN and CSNK1A1 (CK1a).

In this Example 1, we evaluated both forward and inverse MAPPIT assay configurations for detection of lenalidomide- and CC-220-induced binding of proteins to CRBN. HEK293T cells were transfected with a plasmid encoding the MAPPIT receptor fusion (pSEL; CRBN in the forward mode, or any of the tested substrate/prey proteins with the inverse mode), a plasmid encoding the MAPPIT gp130 fusion (CRBN in the inverse mode, or any of the tested substrate/prey proteins in the forward mode) and a STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). Full size proteins were fused for each of the target/prey proteins tested, except in the case of IKZF1 where isoform 7 was used. The MAPPIT receptor fusion applied in this Example 1 consists of the protein of interest (CRBN or target/prey protein) fused to the engineered signaling-deficient cytoplasmic domain of the leptin receptor, which itself is fused to the extracellular domain of the erythropoietin (EPO) receptor. The extracellular EPO receptor domain can be used interchangeably with the extracellular leptin receptor domain (as used in Example 4) to promote receptor/receptor-associated JAK2 activation (with EPO or Leptin, respectively). Cells were treated with erythropoietin (EPO) without or with the indicated dose of test compound at 24 hours after transfection. Luciferase activity was measured 24 hours after test compound treatment using the Luciferase Assay System kit (PROMEGA, Madison, Wis.) with an Ensight plate reader (PERKIN ELMER LIFE SCIENCES, Waltham, Mass.). Data points depict fold induction of the average luciferase activity of triplicate samples from EPO+test compound treated cells versus EPO only treated cells. Error bars represent standard deviations. Curves were fit using 4-parameter nonlinear regression in GRAPHPAD PRISM software. As shown, for most of the CRBN-(neo)substrate/prey interactions, only the inverse assay configuration was able to detect the compound-induced CRBN interactions.

### Example 2: Immunofluorescence Staining of Gp130 Fusions Reveals Exclusively Nuclear Localization of Gp130-IKZF3 Fusion Protein, in Accordance with Lack of Signal in the CRBN-IKZF3 MAPPIT Forward Assay Configuration

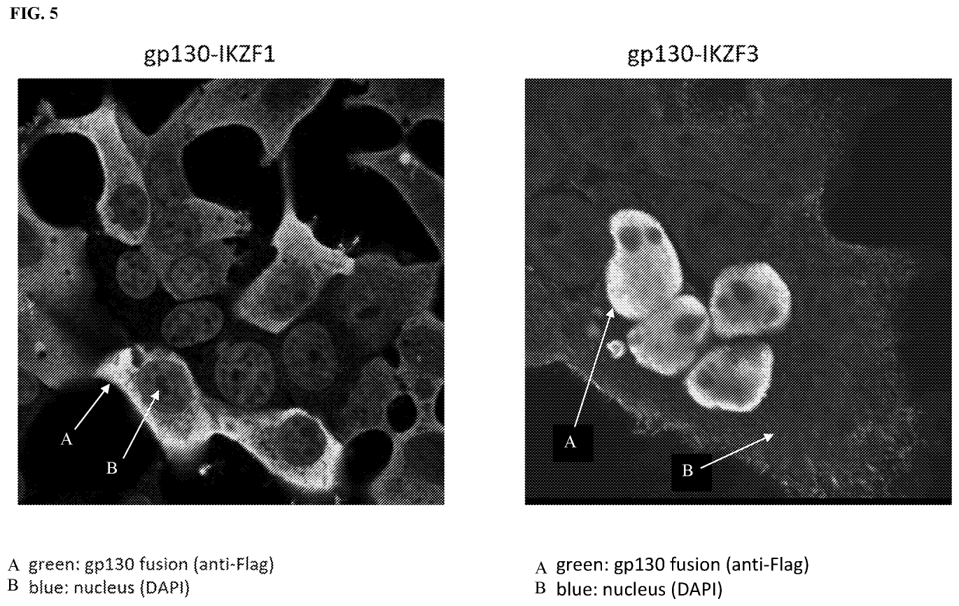

In this study, we evaluated the subcellular localization of gp130 fusion proteins via immunofluorescence staining. As mentioned in Example 1, absence of cytosolic expression of a gp130-target fusion protein can be a reason why a particular interaction is not detected in MAPPIT when using the forward configuration. Therefore, HEK293T cells were seeded on poly-L-lysine coated glass slides and transfected with plasmids encoding Flag-gp130-IKZF3 or Flag-gp130-IKZF1 (isoform7) 24 hours later. Another 24 hours after transfection the cells were fixed with paraformaldehyde, permeabilized with Triton X-100 and stained with anti-Flag (SIGMA) primary and subsequently with a AlexaFluor488-labeled secondary antibody (THERMO Scientific). In parallel, nuclei were stained with DAPI dye (SIGMA). The stained cell preparations were mounted with Vectashield mounting solution (VECTOR Laboratories) and imaged with a confocal microscope (OLYMPUS). The gp130 fusion protein and DAPI staining are shown in green and blue on the images shown in FIG. 5, respectively. The microscopy images obtained clearly indicate the difference in subcellular localization between the IKZF1 and IKZF3 gp130 fusion proteins, expression being restricted to the cytoplasm or the nucleus, respectively, in line with the results obtained in the MAPPIT forward assay configuration, as discussed before.

### Example 3: Non-Specific Binding of CSNK1A1-Gp130 Fusion to MAPPIT Chimeric Receptor Common Component Resulting in High Background Reporter Signal, Obscuring Compound-Dependent Signal Detection in Forward Assay Configuration

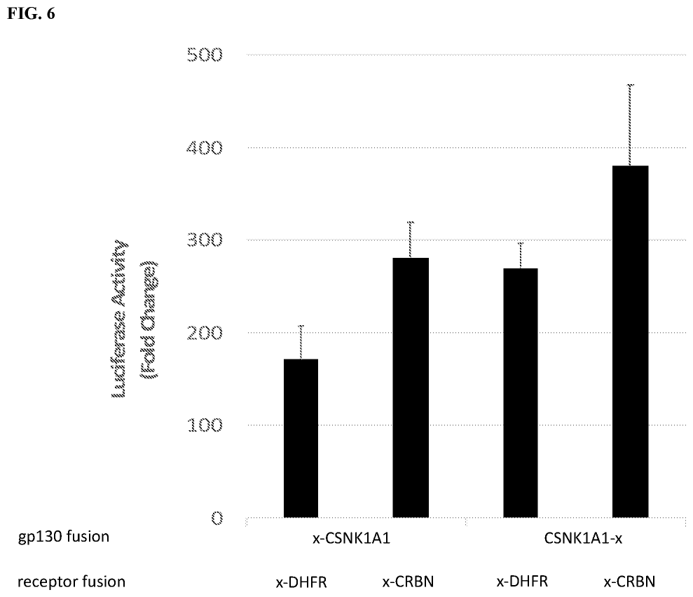

In this Example 3, we used the forward MAPPIT assay for protein-protein interaction analysis, as described in Lemmens, et al. “MAPPIT, a mammalian two-hybrid method for in-cell detection of protein-protein interactions,” Methods Mol Biol. 2015; 1278:447-55. HEK293T cells were transfected with a plasmid encoding the MAPPIT receptor fusion also applied in Example 1 (EPO extracellular domain fused to the engineered cytoplasmic domain of the leptin receptor) linked to either CRBN or E. coli DHFR (dihydrofolate reductase) together with a plasmid encoding a CSNK1A1-gp130 fusion (either N- or C-terminally fused) and a STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). Cells were treated with EPO or left untreated at 24 hours after transfection. Luciferase activity was measured 24 hours later using the Luciferase Assay System kit (PROMEGA, Madison, Wis.) with an Ensight plate reader (PERKIN ELMER LIFE SCIENCES, Waltham, Mass.). Data points depict fold induction of the average luciferase activity of triplicate samples from EPO-treated cells versus untreated cells. Error bars represent standard deviations. The observed strong signals are indicative of a strong interaction occurring of the CSNK1A1-gp130 fusion protein with the chimeric receptor protein, regardless of the nature of the bait protein attached to it, thus binding to a component of the transmembrane chimeric receptor other than the bait protein. As discussed in Example 1, this stickiness hampers CSNK1A1 interaction analysis with CRBN in the forward assay configuration. See FIG. 6.

### Example 4: Evaluation of an Alternative CSNK1A1 Chimeric Receptor Fusion Protein in the MAPPIT Inverse CRBN Interaction Assay

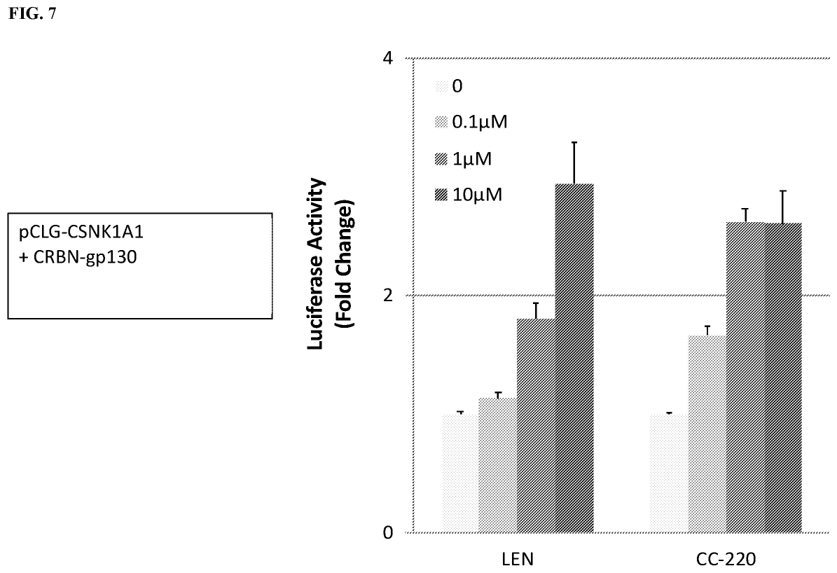

Here, an inverse MAPPIT interaction assay for detection of lenalidomide- and CC-220-dependent interaction between CRBN and the CSNK1A1 neosubstrate was used that is similar to the one discussed in Example 1, but applying an alternative CSNK1A1 receptor fusion. As referred to already in Example 1, alternative receptor fusions are available where the EPO extracellular domain was exchanged for that of the leptin receptor, resulting in the assay system being activated by leptin instead of EPO. In the current example, HEK293T cells were transfected with a plasmid encoding CSNK1A1 fused to a MAPPIT receptor fusion containing the leptin receptor extracellular domain (pCLG-CSNK1A1), a plasmid encoding CRBN fused to the partial gp130 domain and a STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). Cells were treated with leptin without or with the indicated dose of test compound at 24 hours after transfection. Luciferase activity was measured 24 hours after test compound treatment using the Luciferase Assay System kit (PROMEGA, Madison, Wis.) with an Ensight plate reader (PERKIN ELMER LIFE SCIENCES, Waltham, Mass.). Data points depict fold induction of the average luciferase activity of triplicate samples from leptin+test compound treated cells versus leptin only treated cells. Error bars represent standard deviations. The data presented in FIG. 7 indicate that also this alternative MAPPIT receptor fusion enables the detection of molecular glue dependent neosubstrate interactions with CRBN in the inverse assay configuration.

### Example 5: Detection of Compound-Induced FKBP1A (FKBP12) Substrate Interactions in Forward and Inverse MAPPIT Assay Configurations

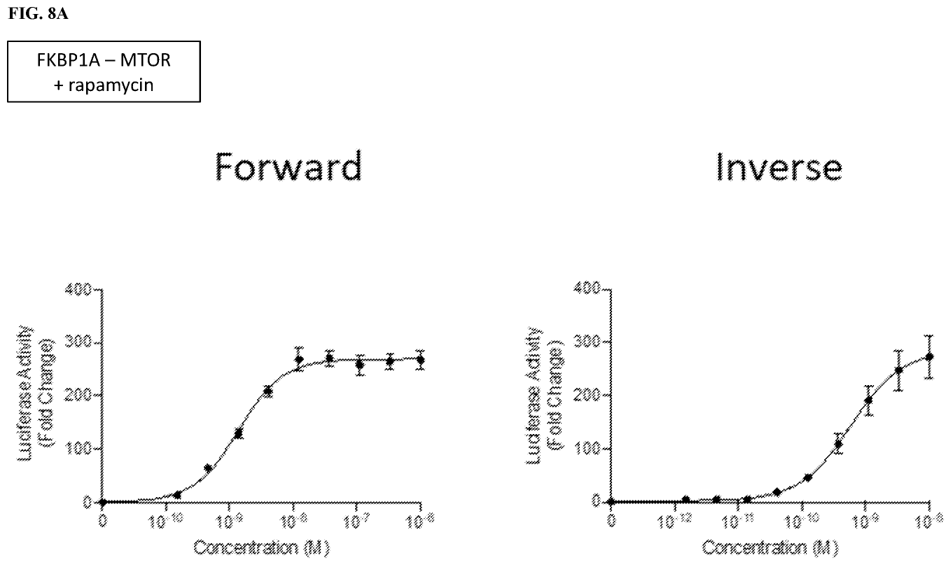

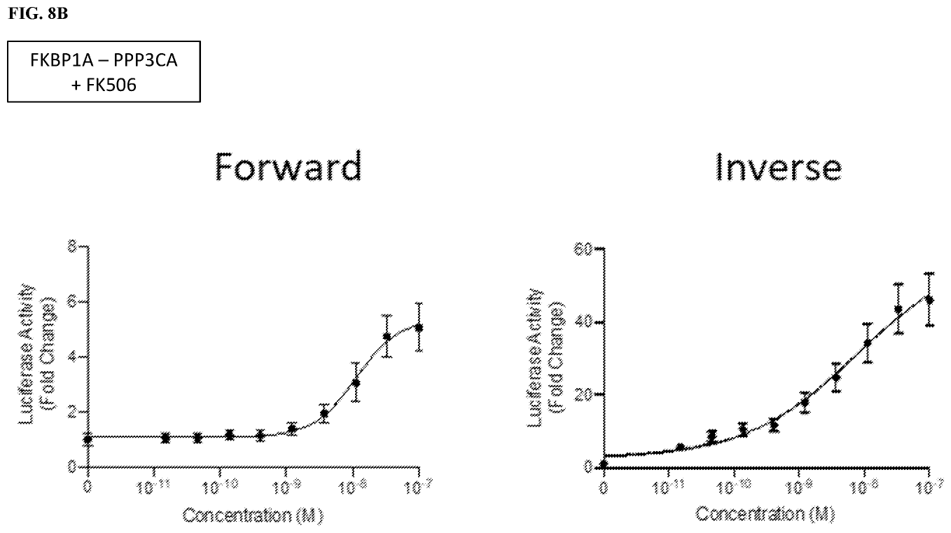

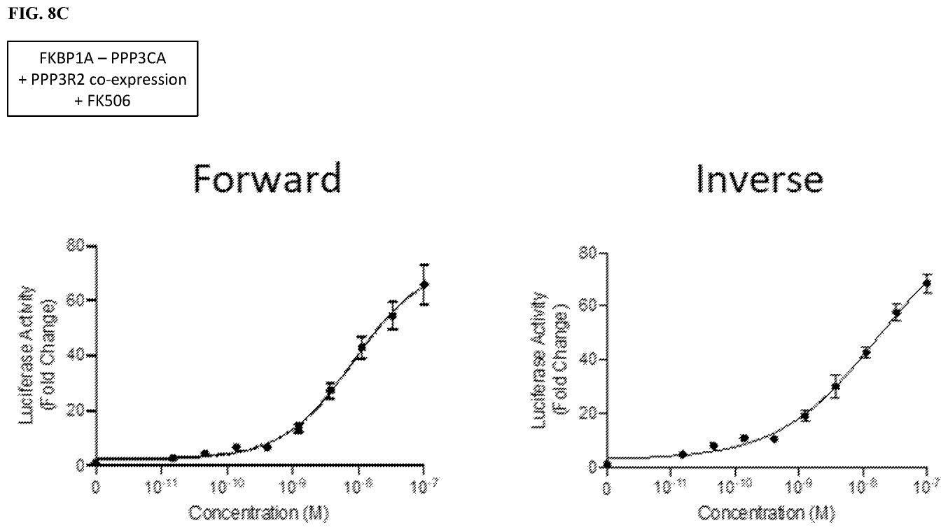

In this Example, forward and inverse MAPPIT assay configurations were compared for the detection of compound-dependent interactions of FKBP1A (FKBP12) with MTOR and calcineurin subunits. The experimental setup was according to the procedure described in Example 1, using the following plasmid constructs encoding the MAPPIT receptor and gp130 fusions: in forward mode the FKBP12 bait was fused to a MAPPIT chimeric receptor construct containing the extracellular EPO receptor domain (pSEL-FKBP1A) and the target proteins were fused to the partial gp130 domain (MTOR FRB domain or PPP3CA); in inverse mode assays, the FKBP1A bait was fused to the partial gp130 domain and MTOR(FRB) or PPP3CA were fused to the MAPPIT transmembrane receptor (pSEL-MTOR(FRB) and pSEL-PPP3CA). For the calcineurin interaction, one additional assay setup was used where in addition to the MAPPIT receptor and gp130 fusions, an unfused PPP3R2-expressing plasmid was co-expressed. PPP3R2 encodes a calcineurin regulatory subunit and has been reported to enhance/promote the FK506 macrolide-induced FKBP1A-calcineurin interaction. HEK293T cells were transfected with the indicated receptor- and gp130-encoding plasmids and a STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). Cells were treated with EPO without or with the indicated dose of test compound (rapamycin or FK506) at 24 hours after transfection. Luciferase activity was measured 24 hours after test compound treatment using the Luciferase Assay System kit (PROMEGA, Madison, Wis.) with an Ensight plate reader (PERKIN ELMER LIFE SCIENCES, Waltham, Mass.). Data points depict fold induction of the average luciferase activity of triplicate samples from EPO+test compound treated cells versus EPO only treated cells. Error bars represent standard deviations. Curves were fit using 4-parameter nonlinear regression in GRAPHPAD PRISM software. The results shown in FIGS. 8A-C indicate that both the forward and inverse assay mode are able to detect the FKBP12 interactions.

### Example 6: Evaluation of Lenalidomide Hybrid Ligand-Induced Binding Between CRBN and DHFR

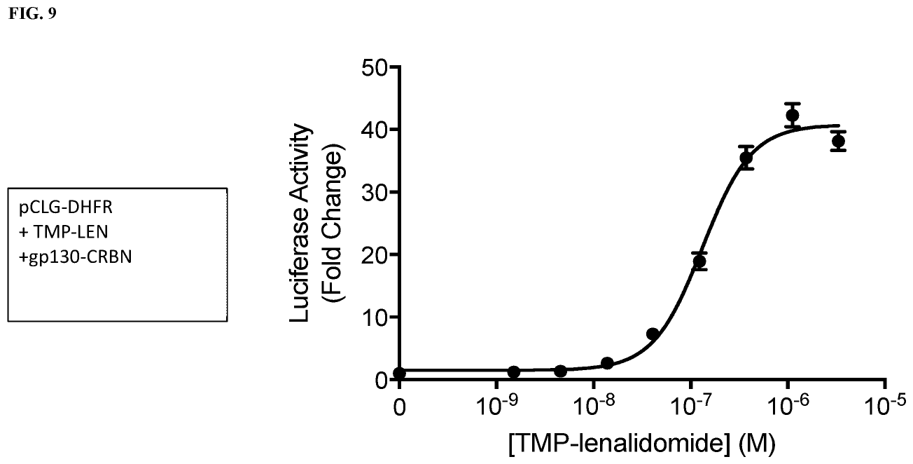

Here, the MAPPIT inverse assay mode was applied to evaluate binding between CRBN and DHFR (dihydrofolate reductase) induced by a hybrid molecule consisting of the DHFR ligand trimethoprim (TMP) fused to the CRBN ligand lenalidomide (LEN) through a PEG linker. HEK293T cells were co-transfected with a plasmid encoding a fusion construct of the (E. coli) DHFR anchor protein tethered to the chimeric MAPPIT-derivative receptor containing the leptin receptor extracellular domain (pCLG-DHFR) and a gp130-CRBN bait fusion construct, together with the STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). At 24 h after transfection the cells were treated with leptin without and with the indicated concentration of TMP-LEN hybrid ligand, and another 24 h later luciferase activity was determined. The dose-response curve shown in FIG. 9 represents the fold induction of the average luciferase activity of triplicate samples from leptin+test compound treated cells versus leptin only treated cells. Error bars represent standard deviations and curves were fit using 4-parameter nonlinear regression in GRAPHPAD PRISM software. This example shows that the MAPPIT inverse assay presented here can be applied to assess binding between two proteins induced by a hybrid ligand.

### Example 7: Characterization of Molecular Glue Binding to CRBN Binding Using a CRBN Inverse MAPPIT Assay

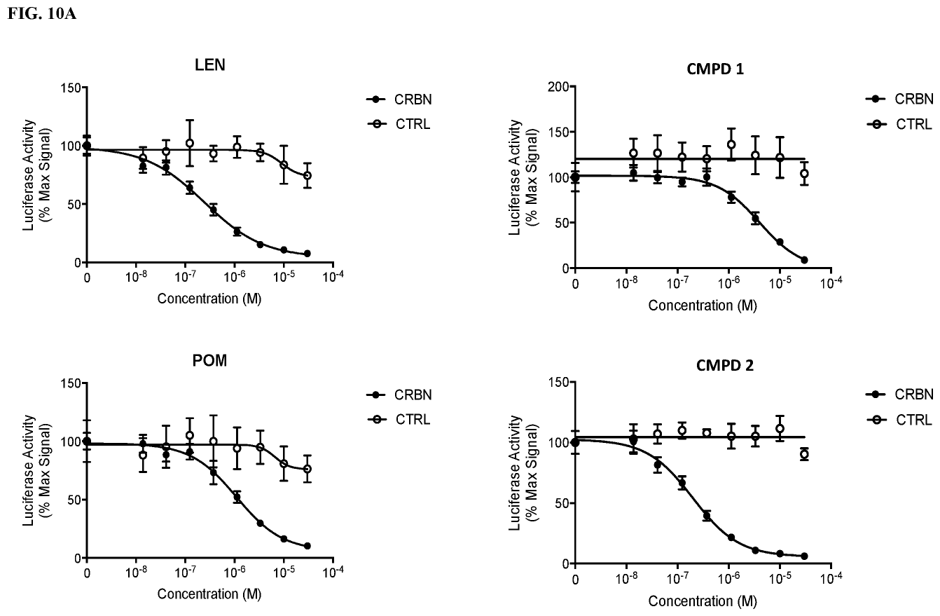

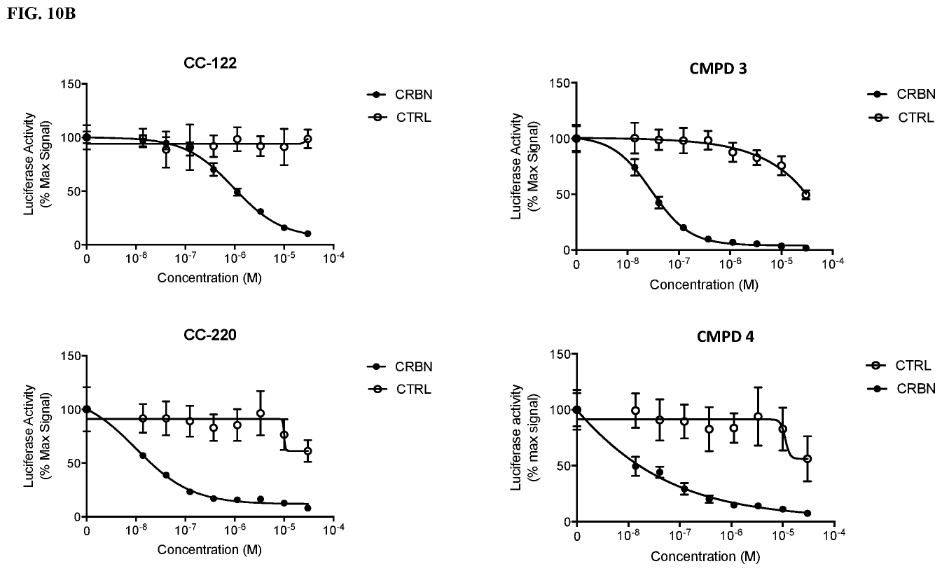

Molecular glue binding to CRBN was assessed with a MAPPIT assay as described in Example 6—by determining the ability of test compounds to compete with a TMP-lenalidomide hybrid ligand for binding to CRBN in cells. As in Example 6, HEK293T cells were transfected with a plasmid encoding E. coli Dihydrofolate Reductase (DHFR) fused to the tails of the cytoplasmic domain of a mutated leptin receptor (pCLG-DHFR), a plasmid encoding a CRBN prey fused to the gp130 cytoplasmic domain or a plasmid encoding a gp130-REM2 control fusion that can directly interact with the leptin receptor of the DHFR fusion protein, and the STAT3 responsive pXP2d2-rPAPI-luciferase reporter plasmid—using a standard transfection method, as described (Lievens, et al. “Array MAPPIT: high-throughput interactome analysis in mammalian cells.” Journal of Proteome Research 8.2 (2009): 877-886). Cells were treated with leptin to activate the leptin receptor fusion protein and supplemented with 300 nM TMP-lenalidomide fusion compound (hybrid ligand, where trimethoprim interacts with DHFR and lenalidomide with CRBN) without or with the indicated dose of test compound at 24 hours after transfection. Luciferase activity, induced by formation of the ternary complex including DHFR-TMP-lenalidomide-CRBN, and consequential activation of STAT3 signaling, was measured 24 hours after compound treatment using the Luciferase Assay System kit (PROMEGA, Madison, Wis.) with an Ensight plate reader (PERKIN ELMER LIFE SCIENCES, Waltham, Mass.). Data points in FIGS. 10A-B represent the average luciferase activity of triplicate samples derived from cells treated with leptin+test compound for the REM2 control (CTRL) or cells treated with leptin+hybrid ligand+test compound (CRBN) relative to leptin (CTRL) or leptin+hybrid ligand (CRBN) only treated samples (the signals obtained in absence of added test compound for both cases is set at 100% of luciferase activity on y-axis). Error bars represent standard deviations. Curves were fit using 4-parameter nonlinear regression in GRAPHPAD PRISM software. As shown in FIGS. 10A-B, known IMiD compounds, such as lenalidomide (LEN), pomalidomide (POM), CC-122 and CC-220 specifically inhibit hybrid-ligand induced luciferase reporter activation in a dose-dependent manner. This reflects effective competition for binding to CRBN and prevention of binding of hybrid ligand to CRBN (hence inhibition of assay signal). In addition, a number of additional compounds were evaluated in this assay (Cmpd1, cmpd2, cmpd3 and cmpd4) and found to specifically inhibit TMP-LEN-induced luciferase signal with varying levels of potency, ranging from micromolar (Cmpd1) to nanomolar (Cmpd4) affinity.

### Example 8: Inverse MAPPIT Detection of TMP-FK506 Hybrid Ligand-Induced Binding Between FKBP1A (FKBP12) and DHFR

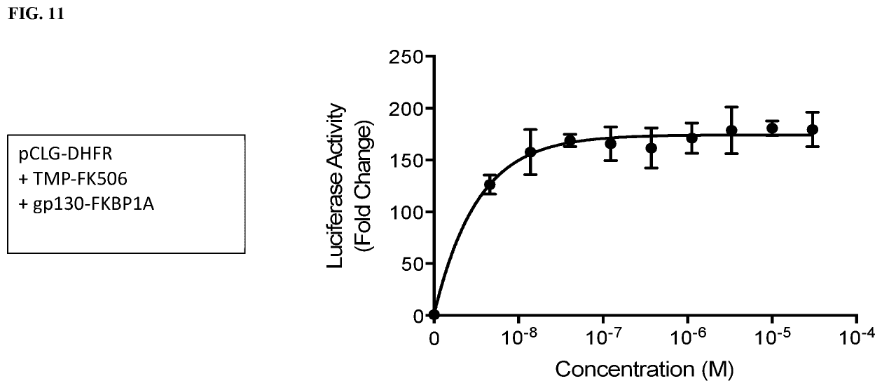

The MAPPIT inverse assay mode was used to test binding between FKBP1A (FKBP12) and DHFR (dihydrofolate reductase) induced by a hybrid molecule consisting of the DHFR ligand trimethoprim (TMP) fused to the FKBP1A ligand FK506 through a PEG linker. HEK293T cells were co-transfected with a plasmid encoding a fusion construct of the (E. coli) DI-FR anchor protein tethered to the chimeric MAPPIT-derivative receptor containing the leptin receptor extracellular domain (pCLG-DHFR) and a gp130-FKBP1A bait fusion construct, together with the STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). At 24 h after transfection the cells were treated with leptin without and with the indicated concentration of TMP-FK506 hybrid ligand, and another 24 h later luciferase activity was determined. The dose-response curve shown in FIG. 11 represents the fold induction of the average luciferase activity of triplicate samples from leptin+test compound treated cells versus leptin only treated cells. Error bars represent standard deviations and curves were fit using 4-parameter nonlinear regression in GRAPHPAD PRISM software. These data indicates that the inverse MAPPIT assay mode also enables detecting hybrid ligand-induced protein recruitment to FKBP1A.

### Example 9: Evaluation of Sulfonamide-Induced Binding of RBM39 to DCAF15 in MAPPIT Forward and Inverse Configuration

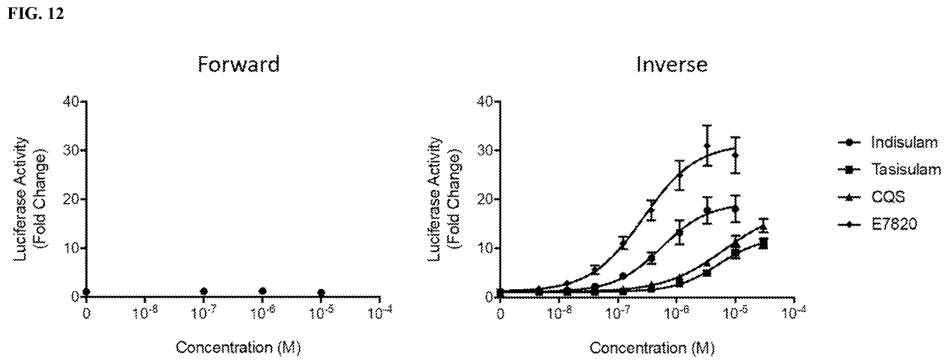

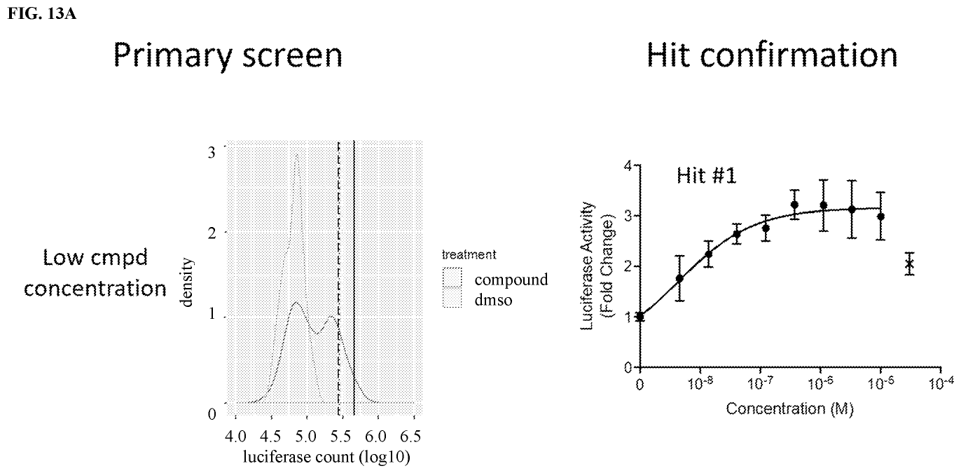

In this example, MAPPIT forward and inverse assays were developed that enable the assessment of sulfonamides to induce binding between DCAF15 and RBM39. DCAF15 is an E3 ligase substrate receptor that has been shown to recruit RBM39 as a substrate for subsequent ubiquitination, and this recruitment is dependent on sulfonamides such as indisulam, tasisulam, chloroquinoxaline sulfonamide (CQS) and E7820. The experimental setup was according to the procedure described before, using the following plasmid constructs encoding the MAPPIT receptor and gp130 fusions: in forward mode the DCAF15 bait was fused to a MAPPIT chimeric receptor construct containing the extracellular leptin receptor domain (pCLL-DCAF15) and RBM39 proteins was fused to the partial gp130 domain; in inverse mode, the DCAF15 bait was fused to the partial gp130 domain and RBM39 was fused to a MAPPIT transmembrane receptor (pCLG-RBM39). HEK293T cells were transfected with the indicated receptor- and gp130-encoding plasmids and a STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). Cells were treated with leptin without or with the indicated dose of test compound (indisulam, tasisulam, CQS or E7820) at 24 hours after transfection. Luciferase activity was measured 24 hours after test compound treatment using the Luciferase Assay System kit (PROMEGA, Madison, Wis.) with an Ensight plate reader (PERKIN ELMER LIFE SCIENCES, Waltham, Mass.). Data points depict fold induction of the average luciferase activity of triplicate samples from leptin+test compound treated cells versus leptin only treated cells. Error bars represent standard deviations. Curves were fit using 4-parameter nonlinear regression in GRAPHPAD PRISM software. The results shown in FIG. 12 indicate that only in the inverse assay mode sulfonamide-induced DCAF15-RBM39 binding can be observed.

### Example 10: Compound Screening for Novel Molecular Glues Inducing Recruitment of SALL4 to CRBN

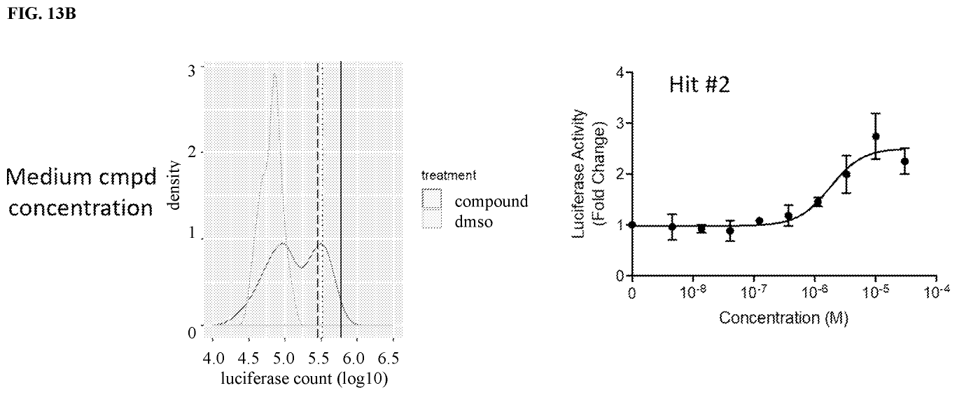

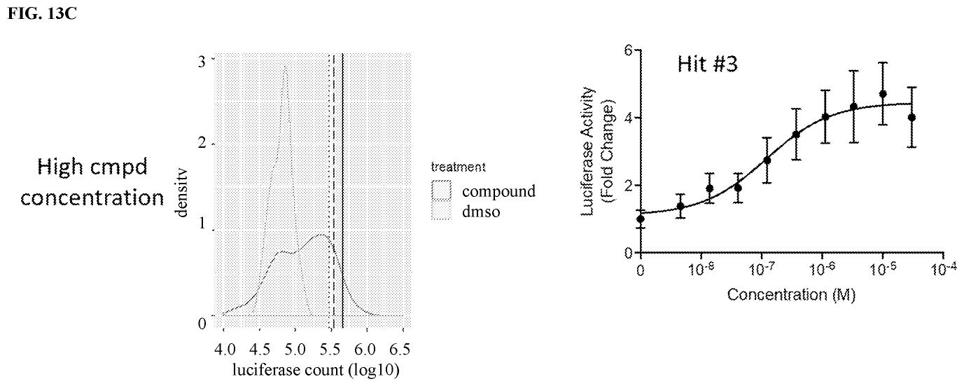

In this example, a compound collection consisting of 96 IMiDs and IMiD-like molecular glues was screened in microtiterplate format to identify compounds that induce recruitment of SALL4 to CRBN, using an inverse MAPPIT CRBN-SALL4 recruitment assay that was also applied in Example 1. HEK293T cells were co-transfected with a plasmid encoding a fusion construct of SALL4 fused to the chimeric MAPPIT membrane receptor containing the EPO receptor extracellular domain (pSEL-SALL4) and a gp130-CRBN bait fusion construct, together with the STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). Cells were treated with EPO and compound (or DMSO as negative control) at 24 hours after transfection. Three concentrations (indicated as ‘low’, ‘medium’ and ‘high’ in FIGS. 13A-C) were applied for each compound: either 0.8, 4 and 20 μM or 0.2, 1 and 5 μM, depending on the previously assessed cellular toxicity level of the compound, and each compound concentration was tested in duplicate. Luciferase activity was measured 24 hours after compound treatment using the Luciferase Assay System kit (PROMEGA, Madison, Wis.) with an Ensight plate reader (PERKIN ELMER LIFE SCIENCES, Waltham, Mass.). The graphs shown in FIGS. 13A-C (left panel) depict the frequency distributions of the average raw luciferase signal for both the compound-treated samples and the DMSO-treated controls, and each graph corresponds with the data for one of the three tested compound concentrations (low, medium and high). The right-shifted portion of the bimodal distribution corresponding to the compound-treated samples represents those compounds with a signal above background and therefore inducing SALL4 recruitment to CRBN. For three such compounds exhibiting a reporter signal above background for one or more of the three tested concentrations, the luciferase signal is indicated by line marks (dotted, dashed or solid) and the corresponding dose-response curves are shown (right panel). These dose-response curves were generated using the same assay setup and protocol used for the primary screen, but now testing a 9-point dose-range of the indicated concentrations. Here, data points depict fold induction of the average luciferase activity of triplicate samples from EPO+test compound treated cells versus EPO only treated cells. Error bars represent standard deviations and curves were fit using 4-parameter nonlinear regression in GRAPHPAD PRISM software. In summary, this example shows that the MAPPIT inverse assay presented here can be applied to screen compound collections to identify known and novel molecular glues inducing substrate recruitment to CRBN. FIGS. 13A-C exemplifies compound screening for glue inducing SALL4 recruitment to CRBN specifically, but the approach can be applied to screen any other potential substrate.

### Example 11: Identification of Novel Molecular Glue-Induced CRBN Substrates Using an Inverse MAPPIT ORF cDNA Library Screening Approach

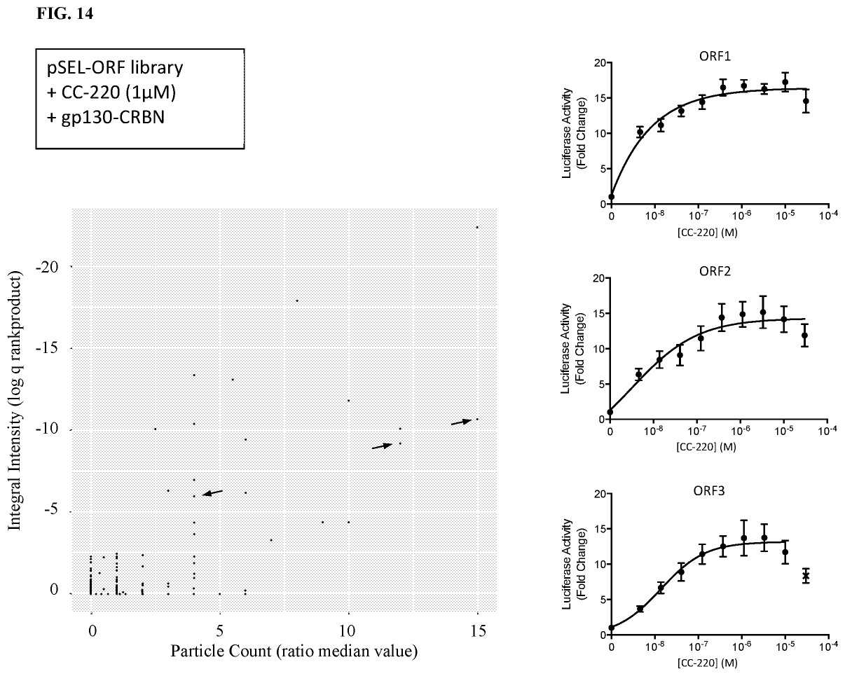

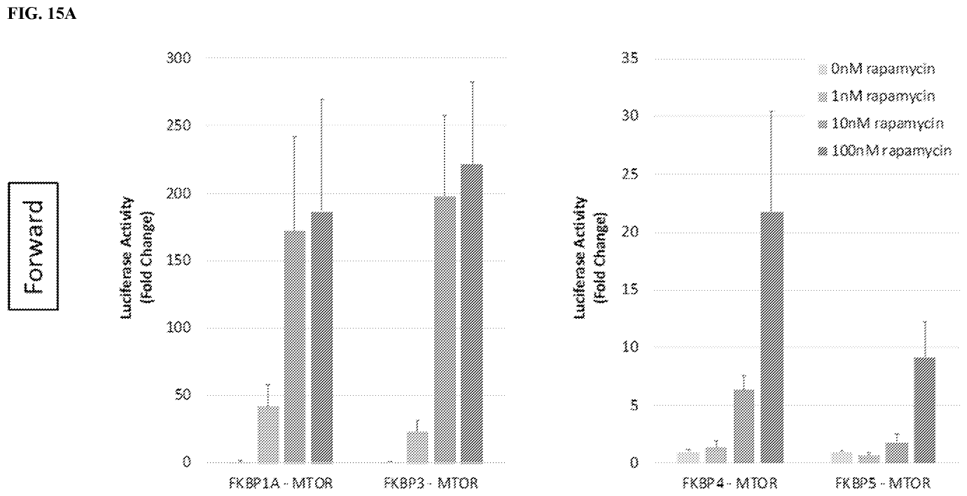

In order to identify ligand-induced CRBN substrates, or neosubstrates, a MAPPIT cell microarray screen was performed using the procedure described in Lievens, et al. “Proteome-scale binary interactomics in human cells.” Molecular & Cellular Proteomics 15.12 (2016): 3624-3639. In brief, HEK293T cells were transfected with a CRBN bait expression plasmid encoding a gp130-CRBN fusion construct. These transfected cells were then added to microarray screening plates containing a MAPPIT chimeric membrane receptor fusion expression plasmid collection covering over 15,000 ORFs. Each spot in the microarray contained a different chimeric receptor-ORF fusion expression plasmid, as well as a STAT3-responsive fluorescence protein-encoding reporter plasmid. Gp130-CRBN bait transfected cells landing and attaching on these spots therefore become transfected as well with the receptor-ORF prey plasmid and the reporter plasmid, resulting in a different CRBN-ORF combination being tested in the cells on every different microarray spot. Twenty-four hours after transfection cells were differentially stimulated with erythropoietin with and without the CRBN ligand CC-220 (1 μM final concentration), and reporter signal (GFP-like fluorescence reporter) was read out 48 hours later. Fluorescence intensity data was analyzed as reported previously, yielding a volcano plot where q-values calculated based on the integrated fluorescence intensity of each microarray cell cluster (Y-axis) are displayed against the ratio of the median value of the fluorescent particle count of the corresponding cell clusters (X-axis), as shown in FIG. 14. Three ORF cDNAs exhibiting a strong signal (indicated by arrows on the dot plot in FIG. 14) were selected for dose-response confirmation using the inverse MAPPIT assay setup: the gp130-CRBN fusion plasmid was co-transfected in HEK293T cells together with the corresponding receptor-ORF plasmid and the luciferase reporter plasmid, 24 h after transfection the cells were treated with EPO without and with the indicated concentration of CC-220, and another 24 h later luciferase activity was determined. The dose-response curves represent the fold induction of the average luciferase activity of triplicate samples from EPO+test compound treated cells versus EPO only treated cells. Error bars represent standard deviations and curves were fit using 4-parameter nonlinear regression in GRAPHPAD PRISM software. As illustrated here for the case of CC-220, this example shows that the inverse MAPPIT assay presented here can be applied to screen ORF cDNA collections to identify known and novel molecular glue-induced CRBN substrates.

### Example 12: Detection of Rapamycin-Induced Recruitment of MTOR to FKBP Proteins in MAPPIT Forward and Inverse Configuration

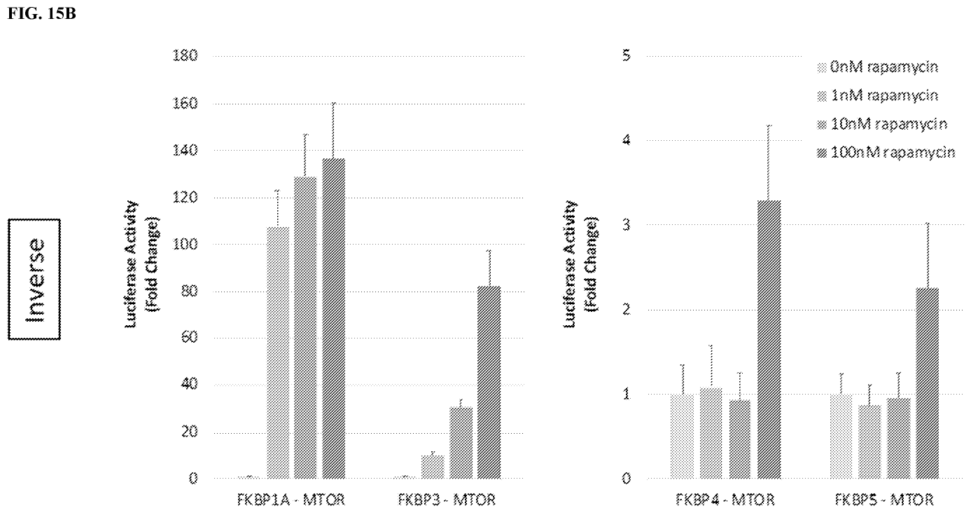

In this Example, MAPPIT forward and inverse assays were developed to monitor rapamycin-induced binding between MTOR and FKBP protein family members, specifically FKBP1A (FKBP12), FKBP3, FKBP4 and FKBP5. In forward mode, the FKBP cDNAs were cloned as MAPPIT receptor fusions containing the EPO receptor extracellular domain (pSEL-FKBPx) and MTOR (FRB domain) was cloned as a gp130 fusion; in inverse mode, the FKBP cDNAs were fused to gp130 and MTOR was cloned as a receptor fusion (pSEL-MTOR). HEK293T cells were co-transfected with a combination of an FKBP fusion construct and an MTOR fusion plasmid and the STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). Cells were treated with EPO without or with the indicated dose of rapamycin at 24 hours after transfection. Luciferase activity was measured 24 hours after test compound treatment using the Luciferase Assay System kit (PROMEGA, Madison, Wis.) with an Ensight plate reader (PERKIN ELMER LIFE SCIENCES, Waltham, Mass.). Data points depict fold induction of the average luciferase activity of triplicate samples from EPO+test compound treated cells versus EPO or leptin only treated cells. Error bars represent standard deviations. As shown in FIGS. 15A-B, both the forward and the inverse MAPPIT assay configurations generated a rapamycin-induced reporter signal for each of the FKBP-MTOR interactions, reproducing published data for these interactions.

What is claimed is:

1. A method for detecting a molecular interaction, comprising:
(a) providing a cell comprising a ligand-dependent chimeric receptor protein comprising:
(i) an extracellular portion of a ligand-binding domain derived from a first receptor and
(ii) transmembrane and cytoplasmic domains of a second receptor and a intracellular prey protein fused thereto, wherein the transmembrane and/or cytoplasmic domains of the second receptor comprise mutations that reduce or eliminate STAT (Signal Transducer and Activator of Transcription) recruitment;
(b) expressing a bait protein that is fused to a receptor fragment in the cell, the receptor fragment comprising functional STAT recruitment sites; and
(c) detecting a signal that is indicative of a molecular interaction,
wherein, the bait protein favors (i) occupying the cytosol over the interior of a membrane bound organelle and/or (ii) specific interaction with the prey protein over non-specific interaction with the cell membrane and/or a non-prey portion of the chimeric receptor.

2. The method of claim 1, wherein the interaction between the prey protein and bait protein causes recruitment of the receptor fragment fused to the bait protein to the transmembrane chimeric receptor protein, which restores ligand-dependent transmembrane chimeric receptor signaling and activation of STAT molecules.

3. The method of claim 2, wherein the cell comprises a STAT-responsive reporter gene.

4. The method of claim 3, wherein the activated STAT molecules migrate to the nucleus and induce transcription of the STAT-responsive reporter gene, the reporter gene signal permitting detection of a molecular interaction.

5. The method of claim 1, wherein the bait protein is not substantially trapped within a cell organelle, optionally selected from cell nucleus, mitochondria, endoplasmic reticulum, and Golgi apparatus.

6. The method of claim 1, wherein the bait protein does not substantially interact with the cellular membrane.

7. The method of claim 1, wherein the bait protein does not substantially interact with the non-prey portion of the chimeric receptor.

8. The method of claim 7, wherein the bait protein does not substantially interact with the transmembrane and/or cytoplasmic domains of the second receptor of the chimeric receptor.

9. The method of any one of the above claims, wherein the bait protein is associated with a scaffold protein, which is optionally fused to the receptor fragment.

10. The method of any one of the above claims, wherein the molecular interaction is a protein/protein interaction.

11. The method of any one of the above claims, wherein the method further comprises introducing a small molecule which binds to the prey protein or bait protein.

12. The method of claim 11, wherein the molecular interaction is a protein/protein interaction which is mediated by the binding of the small molecule with the prey protein or bait protein.

13. The method of any one of the above claims, wherein the molecular interaction is two or more protein/protein interactions which are mediated by the binding of the small molecule with the prey protein or bait protein.

14. The method of any one of claims 11-13, wherein the protein/protein interaction which is mediated by the binding of the small molecule with the prey protein or bait protein is a direct binding between the prey protein or bait protein and the small molecule at a protein/protein interface.

15. The method of any one of claims 11-13, wherein the protein/protein interaction which is mediated by the binding of the small molecule with the prey protein or bait protein is mediated by an allosteric modification of the protein surface of the prey protein or bait protein.

16. The method of claim 15, wherein the small molecule induces exposure of a hydrophobic surface of the prey protein or bait protein that allows for interaction with the prey protein or bait protein.

17. The method of any one of claim 15 or 16, wherein the small molecule induces exposure of a hydrophobic surface of the bait protein that allows for interaction with the prey protein.

18. The method of any one of claim 15 or 16, wherein the small molecule induces exposure of a hydrophobic surface of the prey protein that allows for interaction with the bait protein.

19. The method of any one of claims 11-18, wherein the small molecule is a molecular glue.

20. The method of claim 1, wherein the molecular interaction is a complex formation.

21. The method of claim 1, wherein the molecular interaction is a small molecule/protein interaction.

22. The method of claim 1, wherein the prey protein is bound to a small molecule and the small molecule is connected via a linker to a second small molecule which binds to the bait protein.

23. The method of claim 1, wherein the bait protein is bound to a small molecule and the small molecule is connected via a linker to a second small molecule which binds to the prey protein.

24. The method of any one of claims 1-23, wherein the first receptor and second receptor are the same.

25. The method of any one of claims 1-24, wherein the first receptor and second receptor are different.

26. The method of any one of claims 1-24, wherein the first receptor and/or second receptor is a multimerizing receptor.

27. The method of any one of claims 1-26, wherein the ligand-binding domain is derived from a cytokine receptor.

28. The method of any one of claims 1-26, wherein the ligand-binding domain is derived from a Type 1 cytokine receptor (CR).

29. The method of any one of claims 1-26, wherein the ligand-binding domain is derived from erythropoietin receptor (EpoR) or leptin receptor (LR).

30. The method of claim 29, wherein the transmembrane and cytoplasmic domains are derived from the murine leptin receptor (LR).

31. The method of any one of claims 1-30, wherein the bait is heterologous to the first receptor and/or second receptor fragment.

32. The method of any one of claims 1-31, wherein the cytoplasmic domain comprises a JAK binding site and/or the receptor fragment comprises gp130.

33. The method of any one of claims 1-32, wherein the STAT is selected from STAT1 or STAT3.

34. The method of any one of claims 1-33, wherein the mutations that reduce or eliminate STAT recruitment are to one or more tyrosine phosphorylation sites.

35. The method of any one of claims 1-34, wherein the transmembrane and cytoplasmic domains are derived from the murine leptin receptor (LR) and the mutations are at one or more of positions Y985, Y1077, and Y1138.

36. The method of any one of claims 1-35, wherein the transmembrane and cytoplasmic domains are derived from the murine leptin receptor (LR) and the mutations are Y985F, Y1077F, and Y1138F.

37. The method of any one of claims 1-36, wherein the transmembrane and cytoplasmic domains have functionally equivalent mutations to Y985F, Y1077F, and Y1138F of the murine leptin receptor (LR).

38. The method of any one of claims 1-37, wherein the bait protein comprises a nuclear export sequence (NES).

39. The method of claim 38, wherein the NES has 1-4 hydrophobic residues.

40. The method of claim 39, wherein the hydrophobic residues are leucines.

41. The method of any one of claims 38-40, wherein the NES has the sequence LxxxLxxLxL, where L is a hydrophobic residue and x is any other amino acid.

42. The method of any one of claims 35-38, wherein the NES has the sequence LxxxLxxLxL, where L is a leucine and x is any other amino acid.

43. The method of any one of the above claims, wherein the bait is a E3 ligase substrate binding subunit, optionally selected from cereblon (CRBN) and Von Hippel Lindau (VHL), and optionally is associated with a scaffold protein, optionally selected from damaged DNA binding protein 1 (DDB1), Cullin-4A (CUL4A), and regulator of cullins 1 (ROC1).

44. The method of claim 43, where the bait is contacted with a compound before interaction with the prey protein.

45. The method of claim 44, wherein the compound comprises a glutarimide ring and a phthalimide ring.

46. The method of claim 45, wherein the compound is selected from thalidomide, lenalidomide, pomalidomide, CC-220, CC-122, CC-885, or a derivative or analog thereof.

47. The method of any of the above claims, wherein the method comprises assaying a plurality of cells comprising a ligand-dependent chimeric receptor comprising (i) an extracellular portion of a ligand-binding domain derived from a first receptor and (ii) transmembrane and cytoplasmic domains of a second receptor and a intracellular prey protein fused thereto.

48. The method of claim 47, wherein a single bait protein is expressed in each cell.

49. The method of claim 47, wherein a single bait protein is assayed for a molecular interaction with a plurality of prey proteins.

50. The method of any of the above claims, wherein the method identifies a novel protein/protein interaction.

51. The method of any of the above claims, wherein the method identifies a novel protein/protein interaction which is mediated by the binding of the small molecule with the prey protein or bait protein.

52. The method of any of the above claims, wherein the method identifies a small molecule compound that induces, mediates or stabilizes a protein-protein interaction that comprises the prey protein and bait protein.

53. The method of claim 52, wherein the small molecule compound is a molecular glue or hybrid ligand.

54. The method of any one of claims 1-42 or 47-53, wherein the bait is an FK506 binding protein (FKBP).

55. The method of any one of claims 1-42 or 47-54, wherein the FK506 binding protein (FKBP) is selected from FKBP12, FKBP38 and FKBP52.

56. The method of claim 55, wherein the compound is selected from FK506 (tacrolimus), rapamycin (sirolimus), and cyclosporin A (CsA) or a derivative or analog thereof or a compound that binds to the same FKBP bait binding site as the FK506 (tacrolimus), rapamycin (sirolimus), and cyclosporin A (CsA) or a derivative or analog thereof and in a competitive fashion.


SEQUENCE LISTING (1 sequence)

SEQ ID NO: 1 - protein, 1162 aa, Artificial Sequence
  Synthetic polymer.
MMCQKFYVVLLHWEFLYVIAALNLAYPISPWKFKLFCGPPNTTDDSFLSPAGAPNNASALKGASEAIVEAKFNSSGIYVPELSKTVFHCCFGNEQGQNCSALTDNTEGKTLASVVKASVFRQLGVNWDIECWMKGDLTLFICHMEPLPKNPFKNYDSKVHLLYDLPEVIDDSPLPPLKDSFQTVQCNCSLRGCECHVPVPRAKLNYALLMYLEITSAGVSFQSPLMSLQPMLVVKPDPPLGLHMEVTDDGNLKISWDSQTMAPFPLQYQVKYLENSTIVREAAEIVSATSLLVDSVLPGSSYEVQVRSKRLDGSGVWSDWSSPQVFTTQDVVYFPPKILTSVGSNASFHCIYKNENQIISSKQIVWWRNLAEKIPEIQYSIVSDRVSKVTFSNLKATRPRGKFTYDAVYCCNEQACHHRYAELYVIDVNINISCETDGYLTKMTCRWSPSTIQSLVGSTVQLRYHRRSLYCPDSPSIHPTSEPKNCVLQRDGFYECVFQPIFLLSGYTMWIRINHSLGSLDSPPTCVLPDSVVKPLPPSNVKAEITVNTGLLKVSWEKPVFPENNLQFQIRYGLSGKEIQWKTHEVFDAKSKSASLLVSDLCAVYVVQVRCRRLDGLGYWSNWSSPAYTLVMDVKVPMRGPEFWRKMDGDVTKKERNVTLLWKPLTKNDSLCSVRRYVVKHRTAHNGTWSEDVGNRTNLTFLWTEPAHTVTVLAVNSLGASLVNFNLTFSWPMSKVSAVESLSAYPLSSSCVILSWTLSPDDYSLLYLVIEWKILNEDDGMKWLRIPSNVKKFYIHDNFIPIEKYQFSLYPVFMEGVGKPKIINGFTKDAIDKQQNDAGLYVIVPIIISSCVLLLGTLLISHQRMKKLFWDDVPNPKNCSWAQGLNFQKPETFEHLFTKHAESVIFGPLLLEPEPISEEISVDTAWKNKDEMVPAAMVSLLLTTPDPESSSICISDQCNSANFSGSQSTQVTCEDECQRQPSVKYATLVSNDKLVETDEEQGFIHSPVSNCISSNHSPLRQSFSSSSWETEAQTFFLLSDQQPTMISPQLSFSGLDELLELEGSFPEENHREKSVCYLGVTSVNRRESGVLLTGEAGILCTFPAQCLFSDIRILQERCSHFVENNLSLGTSGENFVPYMPQFQTCSTHSHKIMENKMCDLTV